(19) United States
(12) Patent Application Publication
(10) Pub. No.: US 2009/0258917 A1
(43) Pub. Date: Oct. 15, 2009

(54) Benzoxazoles Useful in the Treatment of Inflammation

(76) Inventors: Benjamin Pelcman, Solna (SE); Kristofer Olofsson, Solna (SE); Wesley Schaal, Solna (SE); Ivars Kalvins, Riga (LV); Martins Katkevics, Riga (LV); Vita Ozola, Riga (LV); Edgars Suna, Riga (LV)

Correspondence Address: MORGAN LEWIS & BOCKIUS LLP, 1111 PENNSYLVANIA AVENUE NW, WASHINGTON, DC, 20004, US

(21) Appl. No.: 12/083,384

(22) PCT Filed: Oct. 12, 2006
(86) PCT No.: PCT/GB2006/003792

§ 371 Date: Jan. 9, 2009

Related U.S. Application Data
(60) Provisional application No. 60/725,301, filed on Oct. 12, 2005.

(51) Int. Cl. A61K 31/423; C07D 263/54; A61P 29/00

(52) U.S. Cl. 514/375; 548/217

(57) ABSTRACT

There is provided the use of a compound of formula I,

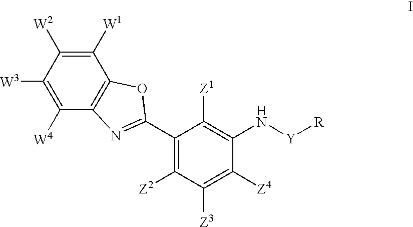

wherein Y, W¹ to W⁴, Z¹ to Z⁴ and R have meanings given in the description, and pharmaceutically-acceptable salts thereof, for the manufacture of a medicament for the treatment of a disease in which inhibition of the activity of a member of the MAPEG family is desired and/or required, and particularly in the treatment of inflammation.

### FIELD OF THE INVENTION

This invention relates to a novel pharmaceutical use of certain compounds, some of which compounds are not known as pharmaceuticals. In particular, this invention relates to the use of such compounds as inhibitors of enzymes belonging to the membrane-associated proteins in the eicosanoid and glutathione metabolism (MAPEG) family. Members of the MAPEG family include the microsomal prostaglandin E synthase-1 (mPGES-1), 5-lipoxygenase-activating protein (FLAP), leukotriene C₄ synthase and microsomal glutathione S-transferases (MGST1, MGST2 and MGST3). Thus, the compounds are of potential utility in the treatment of inflammatory diseases including respiratory diseases.

1. Background of the Invention

There are many diseases/disorders that are inflammatory in their nature. One of the major problems associated with existing treatments of inflammatory conditions is a lack of efficacy and/or the prevalence of side effects (real or perceived).

Inflammatory diseases that affect the population include asthma, inflammatory bowel disease, rheumatoid arthritis, osteoarthritis, rhinitis, conjunctivitis and dermatitis.

Inflammation is also a common cause of pain. Inflammatory pain may arise for numerous reasons, such as infection, surgery or other trauma. Moreover, several diseases including malignancies and cardioavascular diseases are known to have inflammatory components adding to the symptomatology of the patients.

Asthma is a disease of the airways that contains elements of both inflammation and bronchoconstriction. Treatment regimens for asthma are based on the severity of the condition. Mild cases are either untreated or are only treated with inhaled β-agonists which affect the bronchoconstriction element, whereas patients with more severe asthma typically are treated regularly with inhaled corticosteroids which to a large extent are anti-inflammatory in their nature.

Another common disease of the airways with inflammatory and bronchoconstrictive components is chronic obstructive pulmonary disease (COPD). The disease is potentially lethal, and the morbidity and mortality from the condition is considerable. At present, there is no known pharmacological treatment capable of changing the course of the disease.

The cyclooxygenase (COX) enzyme exists in two forms, one that is constitutively expressed in many cells and tissues (COX-1), and one that in most cells and tissues is induced by pro-inflammatory stimuli, such as cytokines, during an inflammatory response (COX-2).

COXs metabolise arachidonic acid to the unstable intermediate prostaglandin H₂ (PGH₂). PGH₂ is further metabolized to other prostaglandins including PGE₂, PGF_{2α}, PGD₂, prostacyclin and thromboxane A₂. These arachidonic acid metabolites are known to have pronounced physiological and pathophysiological activity including pro-inflammatory effects.

PGE₂ in particular is known to be a strong pro-inflammatory mediator, and is also known to induce fever and pain. Consequently, numerous drugs have been developed with a view to inhibiting the formation of PGE₂, including “NSAIDs” (non-steroidal antiinflammatory drugs) and “coxibs” (selective COX-2 inhibitors). These drugs act predominantly by inhibition of COX-1 and/or COX-2, thereby reducing the formation of PGE₂.

However, the inhibition of COXs has the disadvantage that it results in the reduction of the formation of all metabolites downstream of PGH₂, some of which are known to have beneficial properties. In view of this, drugs which act by inhibition of COXs are therefore known/suspected to cause adverse biological effects. For example, the non-selective inhibition of COXs by NSATDs may give rise to gastrointestinal side-effects and affect platelet and renal function. Even the selective inhibition of COX-2 by coxibs, whilst reducing such gastrointestinal side-effects, is believed to give rise to cardiovascular problems.

An alternative treatment of inflammatory diseases that does not give rise to the above-mentioned side effects would thus be of real benefit in the clinic. In particular, a drug that inhibits (preferably selectively) the transformation of PGH₂ to the pro-inflammatory mediator PGE₂ might be expected to reduce the inflammatory response in the absence of a corresponding reduction of the formation of other, beneficial arachidonic acid metabolites. Such inhibition would accordingly be expected to alleviate the undesirable side-effects mentioned above.

PGH₂ may be transformed to PGE₂ by prostaglandin-E synthases (PGES). Two microsomal prostaglandin E synthases (mPGES-1 and mPGES-2), and one cytosolic prostaglandin E synthase (cPGES) have been described.

The leukotrienes (LTs) are formed from arachidonic acid by a set of enzymes distinct from those in the COX/PGES pathway. Leukotriene B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. The biological activities of the CysLTs are mediated through two receptors designated CysLT₁ and CysLT₂. As an alternative to steroids, leukotriene receptor antagonists (LTRas) have been developed in the treatment of asthma. These drugs may be given orally, but do not control inflammation satisfactorily. The presently used LTRas are highly selective for CysLT₁. It may be hypothesised that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs. Among these proteins, 5-lipoxygenase, 5-lipoxygenase-activating protein (FLAP), and leukotriene C₄ synthase may be mentioned. A FLAP inhibitor would also decrease the formation of the proinflammatory LTB₄.

mPGES-1, FLAP and leukotriene C₄ synthase belong to the membrane-associated proteins in the eicosanoid and glutathione metabolism (MAPEG) family. Other members of this family include the microsomal glutathione S-transferases (MGST1, MGST2 and MGST3). For a review, c.f. P. -J. Jacobsson et al in Am. J. Respir. Crit. Care Med 161, S20 (2000). It is well known that compounds prepared as antagonists to one of the MAPEGs may also exhibit inhibitory activity towards other family members, c.f. J. H Hutchinson et al in J. Med Chem. 38, 4538 (1995) and D. Claveau et al in J. Immunol. 170, 4738 (2003). The former paper also describes that such compounds may also display notable cross-reactivity with proteins in the arachidonic acid cascade that do not belong to the MAPEG family, e.g. 5-lipoxygenase.

Thus, agents that are capable of inhibiting the action of mPGES-1, and thus reducing the formation of the specific arachidonic acid metabolite PGE₂, are likely to be of benefit in the treatment of inflammation. Further, agents that are capable of inhibiting the action of the proteins involved in the synthesis of the leukotrienes are also likely to be of benefit in the treatment of asthma and COPD.

2. Prior Art

International patent applications WO 2005/030705, WO 2005/030704, WO 2004/032716, WO 03/045929, WO 03/045930, WO 03/037274 and WO 03/011219, and journal articles Chemistry and Biology (2004), 11 (9), 1293-1299by Kao et al and Biochemistry and Medicinal Chemistry Letters (2004), 14 (6), 1455-1459 by Gong et al all disclose various benzoxazoles, or analogues thereof (e.g. oxazolopyridines) that are useful as pharmaceuticals. However none of these documents suggest the use of such compounds as inhibitors of a member of the MAPEG family, and thus in the treatment of inflammation. International patent applications WO 2004/046122 and WO 2004/046123 disclose benzoxazole derivatives that may be useful as heparanase inhibitors, and thus in the treatment of inflammation. However, the former document does not mention or suggest compounds that are not substituted (via a linker group or otherwise) by a carboxy or tetrazolyl group. Further, the latter document does not mention or suggest benzoxazoles substituted with a phenyl ring, in which that phenyl ring is substituted by an aromatic amido group.

International patent application WO 2004/035522 discloses inter alia benzoxazoles for use as probes for the imaging diagnosis of diseases in which prion protein is accumulated. This document does not mention or suggest the use of the compounds disclosed therein as inhibitors of a member of the MAPEG family, and thus in the treatment of inflammation.

International patent application WO 96/11917 discloses heteroaryl groups including benzoxazoles that may be useful as PDE IV inhibitors, and therefore in the treatment of inflammation. However, there is no disclosure in this document of benzoxazoles that are substituted in the 2-position with two consecutive aromatic groups, nor is there the suggestion of the use of the compounds disclosed therein as inhibitors of a member of the MAPEG family.

International patent application WO 2004/089470 discloses various compounds that may be useful in modulating the activity of 11 β-hydroxysteroid dehydrogenase type 1, for use in, for example, cancer. International applications WO 2004/089416 and WO 2004/089415 also disclose the use of these compounds in combination therapy. However, none of these documents disclose or suggest the use of such compounds as inhibitors of a member of the MAPEG family.

### DISCLOSURE OF THE INVENTION

According to the invention there is provided a use of a compound of formula I,

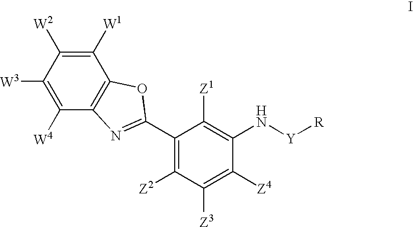

wherein

R represents aryl or heteroaryl, both of which are optionally substituted by one or more substituents selected from X¹;

Y represents —C(O)— or —S(O)₂—;

W¹ to W⁴ and Z¹ to Z⁴ independently represent hydrogen or a substituent selected from X²;

X¹ and X² independently represent halo, —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4c}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p} or —S(O)₂N(R^{4h})R^{5h};

m represents 0, 1 or 2;

R^{3b}, R^{3d} to R^{3h}, R^{3k}, R³ⁿ, R^{4a} to R^{4h}, R^{5a}, Ra^{R}^{5b}, R^{5d} and R^{5f} to R^{5h} independently represent H or R^{3a}; or

any of the pairs R^{4a} and R^{5a}, R^{4b} and R^{5b}, R^{4d} and R^{5d}, R^{4f} and R^{5f}, R^{4g} and R^{5g} or R^{4h} and R^{5h} may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or R^{3al ;}

R^{3c}, R³ⁱ, R^{3j}, R^{3m} and R^{3p} independently represent R^{3a};

R^{3a} represents, on each occasion when mentioned above, C₁₋₆ alkyl optionally substituted by one or more substituents selected from F, Cl ═O, —OR^{6a} or —N(R^{6b})R^{7b};

R^{6a} and R^{6b} independently represent H or C₁₋₆ alkyl optionally substituted by one or more substituents selected from F, Cl, ═O, —OR^{8a}, —N(R^{9a})R^{10a} or —S(O)₂-G¹;

R^{7b} represents H, —S(O)₂CH₃, —S(O)₂CF₃ or C₁₋₆ alkyl optionally substituted by one or more substituents selected from F, Cl, ═O, —OR^{11a}, —N(R^{12a})R^{13a} or —S(O)₂₋—G²; or R^{6b} and R^{7b} may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or C₁₋₃ alkyl optionally substituted by one or more fluoro atoms;

G¹ and G² independently represent —CH₃, —CF₃ or —N(R^{14a})R^{15a};

R^{8a} and R^{11a} independently represent H, —CH₃, —CH₂CH₃ or —CF₃;

R^{9a}, R^{10a}, R^{12a}, R^{13a}, R^{14a} and R^{15a} independently represent H, —CH₃ or —CH₂CH₃,

or a pharmaceutically acceptable salt thereof,

for the manufacture of a medicament for the treatment of a disease in which inhibition or modulation of the activity of a member of the MAPEG family is desired and/or required.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of formula I may contain double bonds and may thus exist as E (entgegen) and Z (zusammen) geometric isomers about each individual double bond. All such isomers and mixtures thereof are included within the scope of the invention.

Compounds of formula I may also exhibit tautomerism. All tautomeric forms and mixtures thereof are included within the scope of the invention.

Compounds of formula I may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl (where q is the upper limit of the range), defined herein may be straight-chain or, when there is a sufficient number (i.e. a minimum of three) of carbon atoms, be branched-chain, and/or cyclic (so forming in the case of alkyl, a C_{3-q} cycloalkyl group). Further, when there is a sufficient number (i.e. a minimum of four) of carbon atoms, such groups may also be part cyclic. Further, unless otherwise specified, such alkyl groups may also be saturated or, when there is a sufficient number (i.e. a minimum of two) of carbon atoms and unless otherwise specified, be unsaturated (forming, for example, a C_{2-q} alkenyl or a C_{2-q} alkynyl group).

The term “halo”, when used herein, includes fluoro, chloro, bromo and iodo.

Aryl groups that may be mentioned include C₆₋₁₄ (e.g. C₆₋₁₀) aryl groups. Such groups may be monocyclic, bicyclic or tricyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an atom of the aromatic ring.

Heteroaryl groups that may be mentioned include those which have between 5 and 14 (e.g. between 5 and 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroaryl groups that may be mentioned include acridinyl, benzimidazolyl, benzodioxanyl, benzodioxepinyl, benzodioxolyl (including 1,3-benzodioxolyl), benzofuranyl, benzofurazanyl, benzothiazolyl, benzothiadiazolyl (including 2,1,3-benzothiadiazolyl), benzoxadiazolyl (including 2,1,3-benzoxadiazolyl), benzoxazinyl (including 3,4-dihydro-2H-1,4-benzoxazinyl), benzoxazolyl, benzimidazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are bicyclic or tricyclic, they are linked to the rest of the molecule via an atom of the aromatic ring. Heteroaryl groups may also be in the N- or S-oxidised form.

Heteroatoms that may be mentioned include include phosphorus, silicon, boron, tellurium, selenium and, preferably, oxygen, nitrogen and sulfur.

For the avoidance of doubt, in cases in which the identity of two or more substituents in a compound of formula I may be the same, the actual identities of the respective substituents are not in any way interdependent For example, in the situation in which W¹ and W² both represent X², then the respective X² groups in question may be the same or different. Similarly, when groups are substituted by more than one substituent as defined herein, the identities of those individual substituents are not to be regarded as being interdependent. For example, when R represents phenyl substituted by R^{3a} and —OR^{3h}, in which R^{3h} represents R^{3a}, and, in each case R^{3a} represents C₁₋₆ alkyl, the identities of the two R^{3a} groups are not to be regarded as being interdependent.

For the avoidance of doubt, when a term such as “W¹ to W⁴”is employed herein, this will be understood by the skilled person to mean W¹, W², W³ and W⁴ inclusively.

Compounds of formula I that may be mentioned include those in which:

Y represents —C(O)—;

when any of the pairs R^{4a} and R^{5a}, R^{4b} and R^{5b}, R^{4d} and R^{5d}, R^{4f} and R^{5f}, R^{4g} and R^{5g} or R^{4h} and R^{5h} are linked together, they together form a 3- to 6-membered ring, which ring optionally contains a further heteroatom (such as nitrogen or oxygen) in addition to the nitrogen atom to which these substituents are necessarily attached, and which ring is optionally substituted by ═O or R^{3a};

R^{3a} represents, on each occasion when mentioned above, C₁₋₆ alkyl optionally substituted by one or more substituents selected from F, Cl, —OCH₃, —OCH₂CH₃ or —OCF₃.

Further, compounds of formula I that may be mentioned include those in which: when Y represents —C(O)—, one of Z¹ to Z⁴ (e.g. Z⁴) represents X², in which X² represents R^{3a}, then R^{3a} represents C₂₋₆ alkyl optionally substituted by one or more substituents selected from F, Cl, —OCH₃, —OCH₂CH₃ or —OCF₃; or when Y represents —C(O)—, one of Z¹ to Z⁴ (e.g. Z⁴) represents X², then X² represents halo, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p} or —S(O)₂N(R^{4h})R^{5h}.

Further compounds of formula I that may be mentioned include those in which: when any one of W¹ to W⁴ (e.g. W² and/or W³) represents X², then X² does not represent —C(O)OR^{3c}; and/or

when any one of W¹ to W⁴ (e.g. W² and/or W³) represents X², then X² does not represent —N(R^{4b})R^{5b} (e.g. when one of R^{4b} and R^{5b} is other than hydrogen).

Preferred compounds of formula I include those in which: when any of the pairs R^{4a} and R^{5a}, R^{4b} and R^{5b}, R^{4d} and R^{5d}, R^{4f} and R^{5f}, R^{4g} and R^{5g} and R^{4h} and R^{5h} are linked together, they form a 5- or 6-membered ring, which ring optionally contains a further heteroatom (such as nitrogen or oxygen) and is optionally substituted by R^{3a} (so forming, for example, a pyrrolidinyl, morpholinyl or apiperazinyl (e.g. 4-methylpiperazinyl) ring);

at least one (such as at least two (e.g. three)) of W¹ to W4 represents hydrogen;

at least one (such as at least two (e.g. three)) of Z¹ to Z⁴ represents hydrogen;

R is substituted with less than four substituents;

X¹ and X² independently represent —S(O)ₘR^{3j}, —N(R^{4b})R^{5b}, —OC(O)R³ⁿ or, more preferably, halo (e.g. bromo, chloro or fluoro), —NO₂, —R^{3a} or —OR³h;

m represents 2;

R^{3a} represents C₁₋₅ alkyl (e.g. difluoromethyl, ethyl, cyclopropyl, t-butyl, cyclopentyl, t-pentyl (i.e. —C(CH₃)₂C₂H₅) or, more preferably, methyl or isopropyl), optionally substituted by one or more fluoro atoms (so forming, for example a trifluoromethyl group);

when R^{3j} represents R^{3a}, then R^{3a} preferably represents C₁₋₃ alkyl (e.g. methyl or ethyl);

when X¹ or X² represents R^{3a}, then R^{3a} preferably represents t-butyl, t-pentyl or, more particularly, methyl or isopropyl, all of which are optionally substituted (and preferably unsubstituted) by one or more halo (e.g. fluoro) atoms (so forming, for example, a trifluoromethyl group);

when R³h represents R^{3a}, then R^{3a} preferably represents cyclopentyl or, particularly, difluoromethyl, ethyl, isbpropyL cyclopropyl, cyclopentyl or, more particularly, methyl or trifluoromethyl;

R^{4b} and R^{5b} independently represent H or methyl; or

R^{4b} and R^{5b} are linked together as herein described;

R³ⁿ represents R^{3a};

when R³ⁿ represents R^{3a}, then R^{3a} preferably represents C₁₋₃ alkyl (e.g. methyl or trifluoromethyl);

R^{6a}, R^{6b} and R^{7b} independently represent H or C₁₋₆ alkyl optionally substituted by one or more fluoro atoms.

Preferred aryl and heteroaryl groups that R may represent include optionally substituted phenyl, naphthyl, pyrrolyl, furanyl, thienyl (e.g. thien-2-yl or thien-3-yl), pyrazolyl, imidazolyl (e.g 1-imidazolyl, 2-imidazolyl or 4-imidazolyl), oxazolyl, isoxazolyl, thiazolyl, pyridyl (e.g. 2-pyridyl, 3-pyridyl or 4-pyridyl), indazolyl, indolyl, indolinyl, isoindolinyl, quinolinyl, 1,2,3,4-tetrahydroqumolinyl, isoquinolinyl, 1,2,3,4-tetrahydroisoquinolinyl, quinolizinyl, benzofuranyL isobenzofuranyl, chromanyl, benzothienyl, pyridazinyl, pyrimidinyl, pyrazinyl (e.g. 2-pyrazinyl), indazolyl, benzimidazolyl, quinazolinyl, quinoxalinyl, 1,3-benzodioxolyl, tetrazolyl, benzothiazolyL and/or benzodioxanyl, group. Preferred values include optionally substituted furanyl, thienyl, oxazolyl, thiazolyl, pyrazinyl (e.g. 2-pyrazinyl) or, more particularly, pyridyl (e.g. 3-pyridyl) or phenyl.

Further preferred compounds of formula I include those in which:

X² represents —OR^{3h}, —N(R^{4b})R^{5b} or, preferably, halo (e.g. fluoro, bromo or, preferably, chloro) or R^{3a};

when X¹ represents R^{3a}, then R^{3a} represents C₁₋₃ alkyl optionally substituted by one or more fluoro substituents;

when X¹ represents —OR^{3h}, then R^{3h} is preferably R^{3a} in which R^{3a} represents C₁₋₃ alkyl optionally substituted by one or more fluoro substituents;

when X² represents R^{3a}, then R^{3a} represents C₁₋₃ alkyl optionally substituted by one or more fluoro substituents;

W¹ to W⁴ independently represent H or a substituent selected from bromo, butyl (e.g. tert-butyl) or, preferably, chloro, methyl and isopropyl;

when one (or two) of W¹ to W⁴ is other than H, then it is preferred that W² and/or W³ is other than H;

Z¹ to Z⁴ independently represent H or a substituent selected from fluoro, —OR^{3h}, —N(R^{4b})R^{5b} or, preferably, chloro and methyl;

when any one of Z¹ to Z⁴ represents —OR^{3h}, then R^{3h} preferably represents H or C₁₋₅ alkyl (e.g. methyl, isopropyl or cyclopentyl);

when any one of Z¹ to Z⁴ represents —N(R^{4b})R^{5b}, then R^{4b} and R^{5b} are independently selected from H or, more preferably, C₁₋₂ alkyl (e.g. methyl) or, R^{4b} and R^{5b} are linked together with the nitrogen atom to which they are attached to form a 4- or, preferably, a 5-membered ring, which ring is preferably unsubstituted and/or preferably contains no further heteroatoms (so forming for example apyrrolidinyl ring);

when one (or two) of Z¹ to Z⁴ is other than H, then it is preferred that it is Z⁴ and/or, more particularly, Z² that is other than H;

when R represents substituted phenyl, then the substituents are preferably selected from amino (e.g. —NH₂) or, preferably, chloro, fluoro, bromo, —NO₂, methyl, trifluoromethyl, methoxy and trifluoromethoxy;

when R represents substituted pyridyl (e.g. 3-pyridyl), then the substituents are preferably selected from fluoro, chloro and trifluoromethyl (and, e.g. in the case of (a) substituent(s) on 3-pyridyl, are preferably in the 2- and/or 6-position).

It is further preferred in compounds of formula I that:

the ring bearing W¹ to W⁴ is substituted by one substituent;

the ring bearing Z¹ to Z⁴ is unsubstituted or substituted by one substituent;

R (e.g. when R is phenyl) is unsubstituted or, more preferably, substituted, for example by one or two substituents, preferably wherein at least one of these substituents is in the ortho position (i.e. resulting in R being substituted in at least in the ortho position), relative to the point of attachment of the R group to the —C(O)— group in the compound of formula I.

Yet more preferred compounds of formula I that may be mentioned include those in which:

W¹ represents H, Cl or methyl;

W² represents H or a substituent as hereinbefore defined (e.g. chloro or, preferably, methyl);

W³ represents H or a substituent as hereinbefore defined (e.g. selected from bromo, tert-hutyl or, preferably, methyl, isopropyl and chloro);

W⁴ represents methyl or, preferably, H;

Z¹ and Z³ independently represent H;

Z⁴ represents a substituent as hereinbefore defined (e.g. methyl) or, more preferably, H;

Z² represents H or, more preferably, a substituent as hereinbefore defined.

Particularly preferred compounds of formula I, or pharmaceuticaUy acceptable salts thereof, include those of the examples described hereinafter.

Compounds of formula I may be made in accordance with techniques that are well known to those skilled in the art, for example as described hereinafter.

According to a further aspect of the invention there is provided a process for the preparation of a compound of formula I, which process comprises:

(i) reaction of a compound of formula II,

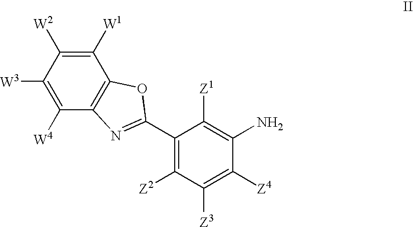

wherein W¹ to W⁴ and Z¹ to Z⁴ are as hereinbefore defined, with a compound of formula III,

R—Y—OH III

wherein R and Y are as hereinbefore defined, under coupling conditions, for example at around room temperature or above (e.g. up to 40-180° C.), optionally in the presence of a suitable base (e.g. sodium hydride, sodium bicarbonate, potassium carbonate, pyrrolidinopyridine, pyridine, triemylamine, tributylamine, trimethylamine, dimethylaminopyridine, diisopropylamine, 1,8-diazabicyclo[5.4.0]undec-7-ene, sodium hydroxide, N-ethyldiisopropylamine, N-(methylpolystyrene)-4-(methylamino)pyridine, butyllithium (e.g. n-, s- or t-butyllithium) or mixtures thereof), an appropriate solvent (e.g. tetrahydrofuran, pyridine, toluene, dichloromethane, chloroform, acetonitrile, dimethylformamide, trifluoromethylbenzene, triethylamine or water) and a suitable coupling agent (e.g. 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino) phosphonium hexafluorophosphate, 2-(1H-benzotriazol-1-yl)-1,1,3,3-tetramethyluronium hexafluorophosphate, benzotriazol-1-yloxytrispyrrolidinophosphonium hexafluorophosphate, bromo-tris-pyrrolidinophosponium hexafluorophosphate, 2-(1H-benzotriazol-1-yl)-1,1,3,3-tetramethyluronium tetrafluorocarbonate) or 1-cyclohexylcarbodiimide-3-propyloxymethyl polystyrene). Alternatively, compounds of formula III may first be activated by treatment with a suitable reagent (e.g. oxalyl chloride, thionyl chloride, etc) optionally in the presence of an appropriate solvent (e.g. dichloromethane, THF, toluene or benzene) and a suitable catalyst (e.g. DMF), resulting in the formation of the respective acyl chloride. This activated intermediate may then be reacted with a compound of formula II under standard conditions, such as those described above. Alternatively, an azodicarboxylate may be employed under Mitsunobo conditions known to those skilled in the art; or

(ii) reaction of a compound of formula IV,

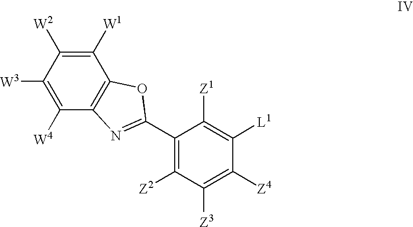

wherein L¹ represents a suitable leaving group, such as chloro, bromo, iodo, a sulfonate group (e.g. —OS(O)₂CF₃, —OS(O)₂CH₃, —OS(O)₂PhMe or a nonaflate) or —B(OH)₂ and W¹ to W⁴ and Z¹ to Z⁴ are as hereinbefore defined, with a compound of formula V,

H₂N—Y—R V

wherein R and Y are as hereinbefore defined, for example optionally in the presence of an appropriate metal catalyst (or a salt or complex thereof) such as Cu, Cu(OAc)₂, CuI (or CuI/diamine complex), Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylenediamine, Na₂CO₃, K₂CO₃, K₃PO₄, Cs₂CO₃, t-BuONa or t-BuOK (or a mixture thereof), in a suitable solvent (e.g. dichloromethane, dioxane, toluene, ethanol, isopropanol, dimethyformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent. This reaction may be carried out at room temperature or above (e.g. at a high temperature, such as the reflux temperature of the solvent system that is employed) or using microwave irradiation.

Compounds of formula II may be prepared by reduction of a compound of formula VI,

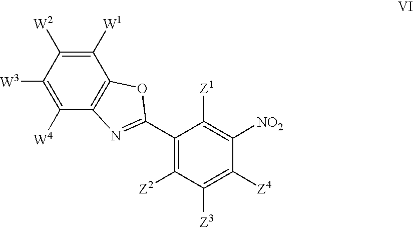

wherein W¹ to W⁴ and Z¹ to Z⁴ are as hereinbefore defined, under standard conditions known to those skilled in the art. For example, the reduction may be performed by hydrogenation (e.g. catalytic hydrogenation (e.g. employing 10% Pd/C)) or in the presence of other suitable reducing conditions, such as employing a mixture of Sn/HCl or Fe powder in EtOH and NH₄Cl.

Compounds of formulae II, IV and VI may be prepared by:

(I) reaction of a compound of formula VII,

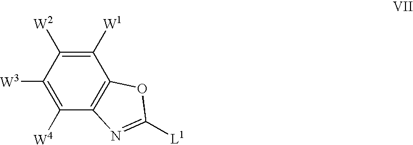

wherein L¹ and W¹ to W⁴ are as hereinbefore defined, with a compound of formula VIII,

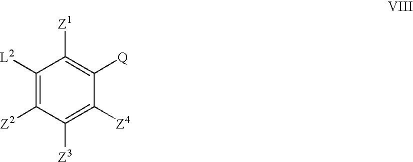

wherein L² represents a suitable leaving group such as chloro, bromo, iodo, —B(OH)₂ or a protected derivative thereof, for example a 4,4,5,5-tetramethyl-1,3,2-dioxaborolan-2-yl group, 9-borabicyclo[3.3.1]nonane (9-BBN), —Sn(alkyl)₃ (e.g. —SnMe₃ or —SnBu₃), or a similar group known to the skilled person, Q represents —NH₂ (for preparation of compounds of formula II), L¹ (for preparation of compounds of formula IV) or —NO₂ (for preparation of compounds of formula VI), as appropriate, and Z¹ to Z⁴ are as hereinbefore defined. The skilled person will appreciate that L¹ and L² will be mutually compatible, and that both must be compatible with Q (e.g. when Q is —NH₂) in compounds of formula VIII. This reaction may be performed, for example in the presence of a suitable catalyst system, e.g. a metal (or a salt or complex thereof) such as CuI, Pd/C, PdCl₂, Pd(OAc)₂, Pd(Ph₃P)₂Cl₂, Pd(Ph₃P)₄, Pd₂(dba)₃ or NiCl₂ and a ligand such as t-Bu₃P, (C₆H₁₁)₃P, Ph₃P, AsPh₃, P(o-Tol)₃, 1,2-bis(diphenylphosphino)ethane, 2,2′-bis(di-tert-butylphosphino)-1,1′-biphenyl, 2,2′-bis(diphenylphosphino)-1,1′-binaphthyl, 1,1′-bis(diphenyl-phosphinoferrocene), 1,3-bis(diphenylphosphino)-propane, xantphos, or a mixture thereof, together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above (e.g. at a high temperature such as the reflux temperature of the solvent system) or using microwave irradiation;

(II) reaction of a compound of formula IX,

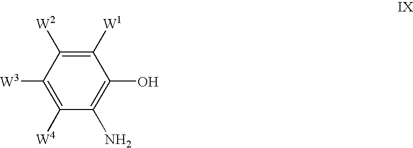

wherein W¹ to W⁴ are as hereinbefore defined, with a compound of formula X,

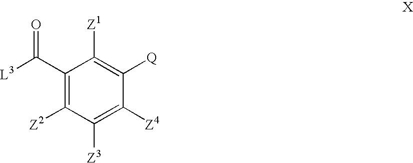

wherem L³ represents a suitable leaving group, such as chloro, bromo, or a hydroxy group, which latter group may be activated by employing a suitable reagent such as one defined hereinbefore in respect of preparation of compounds of formula I (process step (i) above), and Q and Z¹ to Z⁴ are as hereinbefore defined, for example under reaction conditions such as those described hereinbefore in respect of preparation of compounds of formula I (process step (i) above), followed by standard condensation/dehydration conditions. The skilled person will appreciate that this reaction step may proceed via intermediates such as compounds of formula XI or XII described hereinafter;

(III) intramolecular reaction of a compound of formula XI,

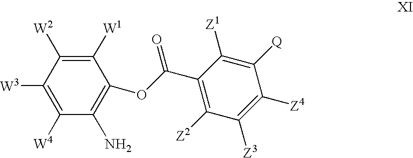

wherein W¹ to W⁴, Z¹ to Z⁴ and Q are as hereinbefore defined or a compound of formula XII,

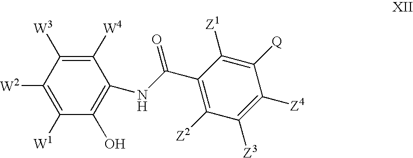

wherein W¹ to W⁴, Z¹ to Z⁴ and Q are as hereinbefore defined, both of which may be allowed to react under reaction conditions known to those skilled in the art, for example standard cyclisation conditions, followed by standard condensation/dehydration conditions; or

(IV) either:
(a) preparing, from a compound of formula VII in which L¹ represents halo:
   (1) a corresponding magnesium-containing reagent (e.g. Grignard reagent) under standard conditions known to those skilled in the art; or
   (2) a corresponding lithiated compound under halogen-lithium exchange reaction conditions known to those skilled in the art; or
(b) preparing, from a compound corresponding to a compound of formula VII but in which L¹ represents H, a compound corresponding to a compound of formula VII but in which L¹ is lithium, under appropriate lithiation conditions,
   and then reacting the resultant intermediate with a compound of formula VIII in which L² represents a suitable leaving group such as bromo, for example under conditions such as those described hereinbefore in respect of preparation of compounds of formulae II, IV or VI (process step (I) above). The skilled person will also appreciate that the magnesium of the magnesium-containing reagent (e.g. Grignard reagent) or the lithium of the lithiated species may be exchanged (and, in the case of the lithiated species, is preferably exchanged) to a different metal (i.e. a transmetallation reaction may be performed), for example to zinc (e.g. using ZnCl₂) and the intermediate so formed may then be subjected to reaction with a compound of formula VIII, for example under reaction conditions described above.

Compounds of formulae III, V, VII, VIII, IX, X, XI, XII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituents W¹ to W⁴, Z¹ to Z⁴ and optional substituents on R in final compounds of formula I or relevant intermediates may be modified one or more times, after or during the processes described above by way of methods that are well known to those skilled in the art. Examples of such methods include substitutions, reductions, oxidations, alkylations, acylations, hydrolyses, esterifications, and etherifications. The precursor groups can be changed to a different such group, or to the groups defined in formula I, at any time during the reaction sequence. In this respect, the skilled person may also refer to “Comprehensive Organic Functional Group Transformations” by A. R. Katritzky, O. Meth-Cohn and C. W. Rees, Pergamon Press, 1995.

For example, in the case where R¹ or R² represents a halo group, such groups may be inter-converted one or more times, after or during the processes described above for the preparation of compounds of formula I. Appropriate reagents include NiCl₂ (for the conversion to a chloro group). Further, oxidations that may be mentioned include oxidations of sulfanyl groups to sulfoxide and sulfonyl groups, for example employing standard reagents (e.g. meta-chloroperbenzoic acid, K₂MnO₄ or a solution of Oxone® in ethylenediaminetetraacetic acid).

Other transformations that may be mentioned include the conversion of a halo group (preferably iodo or bromo) to a cyano or 1-alkynyl group (e.g. by reaction with a compound which is a source of cyano anions (e.g. sodium, potassium, copper (I) or zinc cyanide) or with a 1-alkyne, as appropriate). The latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine). Further, amino groups and hydroxy groups may be introduced in accordance with standard conditions using reagents known to those skilled in the art.

Compounds of formula I may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

Compounds of formula I and salts thereof are useful because they possess pharmacological activity. Such compounds/salts are therefore indicated as pharmaceuticals.

Certain compounds of formula I have not been disclosed before for use as pharmaceuticals. According to a further aspect of the invention there is provided a compound of formula I as hereinbefore defined, or a pharmaceutically-acceptable salt thereof, for use as a pharmaceutical provided that when Y represents —C(O)—, W¹, Z¹ and Z³ all represent hydrogen, and:
(A) W², W³ and W⁴ all represent H, then:
   (i) when Z² represents H and Z⁴ represents —NH₂, then R does not represent 4-(aminoacetyl)phenyl (i.e. 4—(N(H)C(O)CH₃)Ph);
   (ii) when Z² represents chloro and Z⁴ represents H, then R does not represent 4-ethoxy-3-nitrophenyL 3,4,5-trimethoxyphenyl, 3,5-dimethoxyphenyl or 2-methyl-3-nitrophenyl;
   (iii) when Z² and Z⁴ both represent H, then R does not represent 3,5-dinitro-4-methylphenyl;
(B) W² and W⁴ both represent H, then:
   (i) when W³ represents chloro, Z² represents H and Z⁴ represents —CH₃, then R does not represent 2-methoxyphenyl;
   (ii) when W³ represents —CH₃, Z² represents chloro and Z⁴ represents H, then R does not represent unsubstituted phenyl;
(C) W², W³, Z² and Z⁴ all represent H and W⁴ represents —CH₃, then R does not represent 3-methylphenyl;
(D) W³, W⁴ and Z⁴ all represent H, then:
   (i) when W² represents bromo and Z² represents H, then R does not represent unsubstituted phenyl;
   (ii) when W² represents —CH₃ and Z² represents chloro, then R does not represent unsubstituted 3-pyridyl;
(E) W² and W³ both represent —CH₃, W⁴ represents H, Z² represents chloro and Z⁴ represents H, then R does not represent unsubstituted phenyl.

There is further provided a compound of formula I as hereinbefore defined, or a pharmaceutically-acceptable salt thereof, for use as a pharmaceutical, provided that when Y represents —C(O)—, W¹, W², W³, W⁴, Z¹, Z³ and Z⁴ all represent hydrogen, Z² represents chloro, then R does not represent 3-benzyloxyphenyl.

Certain compounds of formula I, and salts thereof, are novel per se. Thus, according to a further aspect of the invention, there is provided:

(I) a compound of formula I (e.g. particularly one in which Y represents —C(O)—) as hereinbefore defined but in which Z³ represents a substituent selected from X², or a pharmaceutically-acceptable salt thereof, provided that when W¹ to W⁴, Z¹, Z² and Z⁴ all represent hydrogen and Z³ represents —CH₃, then R does not represent 4-ethoxyphenyl; and/or

(II) a compound of formula I (e.g. particularly one in which Y represents —C(O)—) as hereinbefore defined but in which any two of Z¹ to Z⁴ represent a substituent selected from X² (and the other two Z¹ to Z⁴ substituents are as hereinbefore defined), or a pharmaceutically-acceptable salt thereof.

There is yet further provided a compound of formula I as hereinbefore defined but in which Y represents —S(O)₂—, or a pharmaceutically-acceptable salt thereof, provided that when W⁴ represents H, Z³ represents H, and:
(A) W¹, W², Z² and Z⁴ all represent H, W³ represents methyl, and:
   (i) Z¹ represents H, then R does not represent unsubstituted phenyl, 4-methylphenyl, 4-(aminoacetyl)phenyl (i.e. 4-(N(H)C(O)CH₃)Ph) or 4-chlorophenyl;
   (ii) Z¹ represents methyl, then R does not represent unsubstituted phenyl;
(B) W¹, W², W³, Z¹ and Z⁴ all represent H, and:
   (i) Z² represents —OH or Cl, then R does not represent unsubstituted phenyl;
   (ii) Z² represents H, then R does not represent unsubstituted phenyl, 4-chlorophenyl, 4-nitrophenyl, 4-(aminoacetyl)phenyl or 4-methylphenyl;
(C) Z⁴ represents methyl and Z¹ and Z² both represent H, and:
   (i) W² represents H and W¹ and W³ both represent methyl; or
   (ii) W² represents methyl and W¹ and W³ both represent H, then (in both cases) R does not represent 2-chloro-5-nitrophenyl;
(D) W², Z¹ and Z² all represent H, and:
   (i) W¹ represents H, W³ represents ethyl or chloro and Z⁴ represents methyl or H; or
   (ii) W¹ and W³ represent chloro and Z⁴ represents methyl, then (in both cases) R does not represent unsubstituted phenyl.

Although compounds of formula I and salts thereof may possess pharmacological activity as such, certain phannaceutically-acceptable (e.g. “protected”) derivatives of compounds of formula I may exist or be prepared which may not possess such activity, but may be administered parenterally or orally and thereafter be metabolised in the body to form compounds of formula I. Such compounds (which may possess some pharmacological activity, provided that such activity is appreciably lower than that of the “active” compounds to which they are metabolised) may therefore be described as “prodrugs” of compounds of formula I.

By “prodrug of a compounds of formula I”, we include compounds that form a compounds of formula I, in an experimentally-detectable amount, within a predetermined time (e.g. about 1 hour), following oral or parenteral administration. All prodrugs of the compounds of formula I are included within the scope of the invention.

Furthermore, certain compounds of formula I may possess no or minimal pharmacological activity as such, but may be administered parenterally or orally, and thereafter be metabolised in the body to form compounds of formula I that possess pharmacological activity as such. Such compounds (which also includes compounds that may possess some pharmacological activity, but that activity is appreciably lower than that of the “active” compounds of formula I to which they are metabolised), may also be described as “prodrugs”.

Thus, the compounds of formula I and salts thereof are useful because they possess pharmacological activity, and/or are metabolised in the body following oral or parenteral administration to form compounds which possess pharmacological activity.

Compounds of formula I and salts thereof are particularly useful because they may inhibit the activity of a member of the MAPEG family.

Compounds of formula I and salts thereof are particularly useful because they may inhibit (for example selectively) the activity of prostaglandin E synthases (and particularly microsomal prostaglandin E synthase-1 (mPGES-1)), i.e. they prevent the action of mPGES-1 or a complex of which the mPGES-1 enzyme forms a part, and/or may elicit a mPGES-1 modulating effect, for example as may be demonstrated in the test described below. Compounds of formula I thereof may thus be useful in the treatment of those conditions in which inhibition of a PGES, and particularly mPGES-1, is required

Compounds of formula I, and pharmaceutically acceptable salts thereof, are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of formula I and salts thereof may be useful in the treatment of asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and ADDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component

Compounds of formula I, and pharmaceutically acceptable salts thereof, may also have effects that are not linked to inflammatory mechanisms, such as in the reduction of bone loss in a subject Conditions that may be mentioned in this regard include osteoporosis, osteoarthritis, Paget's disease and/or periodontal diseases. Compounds of formula I and pharmaceutically acceptable salts thereof may thus also be useful in increasing bone mineral density, as well as the reduction in incidence and/or healing of fractures, in subjects.

Compounds of formula I are indicated both in the therapeutic and/or prophylactic treatment of the above-mentioned conditions.

According to a further aspect of the present invention, there is provided a method of treatment of a disease which is associated with, and/or which can be modulated by inhibition of a member of the MAPEG family, such as a PGES (e.g. mPGES-1), LTC₄ and/or FLAP and/or a method of treatment of a disease in which inhibition of the activity of a member of the MAPEG family, such as PGES (and particularly mPGES-1), LTC₄ and/or FLAP is desired and/or required (e.g. inflammation), which method comprises administration of a therapeutically effective amount of a compound of formula I, or a pharmaceutically acceptable salt thereof, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of formula I will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of formula I may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the formula I, as specified herein, or a pharmaceutically acceptable salt thereof, in admixture with a pharmaceutically acceptable adjuvant, diluent or carrier.

The invention further provides a process for the preparation of a pharmaceutical formulation, as hereinbefore defined, which process comprises bringing into association a compound of formula I, as specified herein, or a pharmaceutically acceptable salt thereof with a pharmaceutically-acceptable adjuvant, diluent or carrier.

Compounds of the formula I may also be combined with other therapeutic agents that are useful in the treatment of inflammation (e.g. NSATDs and coxibs).

According to a further aspect of the invention, there is provided a combination product comprising:
(A) a compound of formula I or a pharmaceutically acceptable salt thereof; and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of formula I, or a pharmaceutically acceptable salt thereof, and the other therapeutic agent).

Thus, mere is further provided:

(1) a pharmaceutical formulation including a compound of formula I or a pharmaceutically acceptable salt thereof, another therapeutic agent that is useful in the treatment of inflammation, and a pharmaceutically-acceptable adjuvant, diluent or carrier; and

(2) a kit of parts comprising components:
(a) a pharmaceutical formulation including a compound of formula I or a pharmaceutically acceptable salt thereof in admixture with a pharmaceutically-acceptable adjuvant, diluent or carrier, and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

The invention further provides a process for die preparation of a combination product as hereinbefore defined, which process comprises bringing into association a compound of formula I or a pharmaceutically acceptable salt thereof with another therapeutic agent that is useful in the treatment of inflammation, and a pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the formula I and salts thereof may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual . instances where higher or lower dosage ranges are merited, and such are within the scope of this invention.

Compounds of formula I and salts thereof may have the advantage that they are effective, and preferably selective, inhibitors of a member of MAPEG family, e.g. inhibitors of the prostaglandin E synthases (PGES) and particularly microsomal prostaglandin E synthase-1 (mPGES-1). The compounds of formula I and salts thereof may reduce the formation of the specific arachidonic acid metabolite PGE₂without reducing the formation of other COX generated arachidonic acid metabolites, and thus may not give rise to the associated side-effects mentioned hereinbefore

Compounds of formula I and salts thereof may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

In the assay mPGES-1 catalyses the reaction where the substrate PGH2 is converted to PGE₂. mPGES-1 is expressed in E. coli and the membrane fraction is dissolved in 20 mM NaPi-buffer pH 8.0 and stored at −80° C. In the assay mPGES-1 is dissolved in 0.1M KPi-buffer pH 7.35 with 2.5mM glutathione. The stop solution consists of H2O/MeCN (7/3), containing FeCl₂ (25 mM) and HCl (0.15 M). The assay is performed at room temperature in 96-well plates. Analysis of the amount of PGE₂ is performed with reversed phase HPLC (Waters 2795equipped with a 3.9×150 mm C18 column). The mobile phase consists of H₂O/MeCN (7/3), containing TFA (0.056%), and absorbance is measured at 195 nm with a Waters 2487 UV-detector.

The following is added chronologically to each well:
1. 100 μL mPGES-1 in KPi-buffer with glutathione. Total protein concentration: 0.02 mg/mL.
2. 1 μL inhibitor in DMSO. Incubation of the plate at room temperature for 25 minutes.
3. 4 μL of a 0.25 mM PGH₂ solution. Incubation of the plate at room temperature for 60 seconds.
4. 100 μL stop solution.
180 μL per sample is analyzed with HPLC.

### EXAMPLES

The invention is illustrated by way of the following examples.

### Example 1

### 4-Isopropyl-N-[3-(5-methylbenzoxazol-2)phenyl]benzamide

(a) 5-Methyl-2-(3-nitrophenyl)benzoxazole

A mixture of 2-amino-4-methylphenol (18 mmol, 2.22 g), 3-nitrobenzoyl chloride (20 mmol, 3.71 g) and 25 mL dioxane (25 mL) was divided into 10 portions, each of which was heated with microwave irradiation for 15 min at 210° C. After cooling, the mixtures were poured into to a stirred solution of NaOH (aq, 1M, 300mL). The yellow precipitate was filtered off, washed with water and dried to afford the sub-title compound (3.03 g, 84%).

(b) 3 -(5 -Methylbenzoxazol-2-phenylamine

A solution of methyl-2-(3-nitro-phenyl)benzoxazole (3.03 g, 11.9 mmol; see step (a) above) in glacial acetic acid (75 mL) was hydrogenated at 4 atm in the presence of 10% Pd-C (127 mg, 1.19 mmol) at rt for 4 h. The mixture was filtered through Celite® and concentrated. The residue was dissolved in EtOAc (100 mL). The solution was washed with NaHCO₃ (aq, sat), dried (Na₂SO₄), filtered through silica gel and concentrated to give the sub-title compound (2.56 g, 96%).

(c) 4-Isopropyl-N-[3-(5-methylbenzoxazol-2-yl)phenyl]benzamide

A mixture of 3-(5-meyhylbenzoxazol-2-yl)phenylamine (560 mg, 2.5 mmol) and 4-isopropylbenzoyl chloride (685 mg, 3.75 mmol) and toluene (25 mL) was heated under reflux for 1.5 h, cooled, filtered and concentrated. The solid was recrystallised from EtOH to afford 355 mg of the title compound. The mother liquor was concentrated and the residue recrystallised from EtOH to yield an additional crop (356 mg). Total yield: 711 mg (77%).

200 MHz ¹H-NMR (DMSO-d₆, ppm) δ 10.4 (1H, s) 8.75 (1H, dd, J=1.6, 1.6 Hz) 8.03-7.87 (4H, m) 769-7.52 (3H, m) 7.44-7.39 (2H, m) 7.27-7.22 (1H, m) 2.98 (1H, septet, J=6.9 Hz) 2.44 (3H, s) 1.24 (6H, d, J=6.9 Hz).

### Example 2

### 3.5-Dichloro-N-[3-(5-methylbenzoxazol-2-yl)phenyl]benzamide

The title compound was prepared in accordance with Example 1, step (c) from 3-(5-methylbebzoxazol-2-yl)phenylamine (see Example 1, step (b)) and 3,5-dichlorobenzoyl chloride.

200 MHz ¹H-NMR (DMSO-d₆, ppm) δ 10.7 (1H, s) 8.70 (1H, dd, J=1.6, 1.6 Hz) 8.03-7.87 (5H, m) 7.66 (1H, d, J=8.4 Hz) 7.63-7.55 (2H, m) 7.24 (1H, dd, J=8.4, 1.6 Hz) 2.43 (3H, s).

### Example 3

### N-[3-(5-Methylbenzoxazol-2-yl)phenyl]-2-nitrobenzamide

The title compound was prepared in accordance with Example 1, step (c) from 3-(5-methylbenzoxazol-2-yl)phenylamine (see Example 1, step (b)) and 2-nitrobenzoyl chloride.

200 MHz ¹H-NMR (DMSO-d₆, ppm) δ 10.95 (1H, s) 8.69-8.67 (1H, m) 8.17 (1H, d, J=8.1 Hz) 7.97-7.89 (2H, m) 7.88-7.83 (3H, m) 7.67 (1H, d, J=8.4 Hz) 7.64-7.53 (2H, m) 7.24 (1H, dd, J=8.1, 1.5 Hz) 2.43 (3H, s).

### Example 4

### 2-Chloro-5-methanesulfonyl-N-[3-(5-methylbenzoxazol-2-yl)phenyl]benzamide

(a) 2-Chloro-5-methanesulfonylbenzoic acid

2-Chloro-5-methylsulfanylbenzoic acid (12.1 g, 59.5 mmol) was suspended in NaOH (aq, 0.5 M, 150 mL). Solid NaHCO₃ (40 g, 480 mmol) followed by acetone (50 mL) was added. After stirring for 5 min at room temperature, a solution of Oxone® (48.5 g) in ethylenediaminetetraacetic acid (aq, 0.0004 M, 180 mL) was added and the mixture was stirred for another 1 h. A solution of NaHSO₃ (30 g, 288 mmol) in water (60 mL) was added with stirring. After 15 min, HCl (aq, 6M, 90 mL) was added. The mixture was extracted with EtOAc and the extract washed with NaHCO₃ (aq, sat), dried, and filtered through silica gel. Concentration gave a solid which was recrystallised from EtOAc/petroleum ether to yield the sub-title compound (11.4 g, 82%).

(b) 2-Chloro-5-methanesulfonylbenzoyl chloride

SOCl₂ (10 mL, 137 mmol), followed by DMF (2 drops) was added to a solution of 2-chloro-5-methanesulfonylbenzoic acid (2.15 g, 9.2 mmol; see step (a) above) in toluene (20 mL). The mixture was heated at reflux for 4 h, cooled and concentrated. The residue was washed several times with dry petroleum ether to afford the crude sub-title compound (2.33 g, 99%) which was used without further purification.

(c) 2-Chloro-5-methanesulfonyl-N-[3-(5-methylbenzoxazol-2-yl)phenyl]benzamide

The title compound was prepared in accordance with Example 1, step (c) from 3-(5-menthylbenzoxazol-2-yl)phenylamine (see Example 1, step (b)) and 2-chloro-5-methanesulfonylbenzoyl chloride (see step (b) above).

200 MHz ¹H-NMR (DMSO-d₆, ppm) δ 10.99 (1H, s) 8.73-8.68 (1H, m) 8.20 (1H, d, J=2.2 Hz) 8.04 (1H, dd, J=8.4, 2.2 Hz) 7.98-7.86 (2H, m) 7.85-7.78 (1H, m) 7.67 (1H, d, J=8.4 Hz) 7.64-7.54 (2H, m) 7.28-7.20 (1H, m) 3.31 (3H, s) 2.43 (3H, s).

### Example 5

### 4-Methanesulfonyl-N-[3-(5-methylbenzoxazol-2-yl]benzamide

The title compound was prepared in accordance with Example 1, step (c) from 3-(5-methylbenzoxazol-2-yl)phenylamine (see Example 1, step (b)) and 4-methanesulfonylbenzoyl chloride.

200 MHz ¹H-NMR (DMSO-d₆, ppm) δ 10.75 (1H, s) 8.77-8.73 (1H, m) 8.26-8.19 (2H, m) 8.14-8.06 (2H, m) 8.04-7.90 (2H, m) 7.67 (1H, d, J=8.4 Hz) 7.64-7.54 (2H, m) 7.24 (1H, dd, J=8.4, 1.1 Hz) 3.29 (3H, s) 2.43 (3H, s).

### Example 6

### 4-Isopropoxy-N-[3-(5-methylbenzoxazol-2-yl)phenyl]benzamide

The title compound was prepared in accordance with Example 1, step (c) from 3-(5-methylbenzoxazol-2-yl)phenylamine (see Example 1, step (b)) and 4-isopropoxybenzoyl chloride.

200 MHz ¹H-NMR (DMSO-d₆, ppm) δ 10.33 (1H, s) 8.77-8.74 (1H, m) 8.03-7.94 (3H, m) 7.91-7.85 (1H, m) 7.67 (1H, d, J=8.4 Hz) 762-7.50 (2H, m) 7.24 (1H, dd, J=8.4, 1.1 Hz) 7.09-7.00 (2H, m) 4.74 (1H, septet, J=5.9 Hz) 2.44 (3H, s) 1.30 (6H, d, J=5.9Hz).

### Example 7

### 3-Isopropoxy-N-[3-(5-methylbenzoxazol-2-yl)phenyl]benzamide

The title compound was prepared in accordance with Example 1, step (c) from 3-(5-methylbenzoxazol-2-yl)phenylamine (see Example 1, step (b)) and 3-isopropoxybenzoyl chloride.

200 MHz ¹H-NMR (DMSO-d₆, ppm) δ 10.44 (1H, s) 8.76-8.72 (1H, m) 8.05-7.96 (1H, m) 7.94-7.86 (1H, m) 7.67 (1H, d, J=8.1 Hz) 7.62-7.50 (4H, m) 7.49-7.38 (1H, m) 7.28-7.20 (1H, m) 7.19-7.11 (1H, m) 4.72 (1H, septet, J=6.2 Hz) 2.43 (3H, s) 1.30 (6H, d, J=6.2) .

### Example 8

### 6-Chloro-N-[3-(5-methylbenzoxazol-2-yl)phenyl]nicotinamide

The title compound was prepared in accordance with Example 1, step (c) from 3-(5-methyllbenzoxazol-2-yl)phenylamine (see Example 1, step (b)) and 6-chloronicotinoyl chloride.

200 MHz ¹H-NMR (DMSO-d₆, ppm) δ 10.73 (1H, s) 8.99 (1H, d, J=2.6 Hz) 8.73-8.69 (1H, m) 8.39 (1H, dd, J=8.4, 2.6 Hz) 8.01-7.89 (2H, m) 7.73 (1H, d, J=8.4Hz) 7.67 (1H, d, J=8.4 Hz) 7.64-7.54 (2H, m) 7.24 (1H, dd, J=8.4, 1.5 Hz) 2.43 (3H,s).

### Example 9

### 3,4-Dimethoxy-N-[3-(5-methylbenzoxazol-2-yl)phenyl]benzamide

The title compound was prepared in accordance with Example 1, step (c) from 3-(5-methylbenzoxazol-2-yl)phenylamine (see Example 1, step (b)) and 3,4-dimethoxybenzoyl chloride.

200 MHz ¹H-NMR (DMSO-d₆, ppm) δ 10.32 (1H, s) 8.72-8.68 (1H, m) 8.06-7.98 (1H, m) 7.92-7.85 (1H, m) 7.71-7.63 (2H, m) 7.61-7.51 (3H, m) 7.28-7.20 (1H, m) 7.10 (1H, d, J=8.4 Hz) 3.85 (3H, s) 3.83 (3H, s) 2.43 (3H, s).

### Example 10

### 2-Chloro-N-[3-(5-methylbenzoxazol-2-yl)phenyl]nicotinamide

The title compound was prepared in accordance with Example 1, step (c) from 3-(5-methylbenzoxazol-2-yl)phenylamine (see Example 1, step (b)) and 2-chloronicotinoyl chloride.

200 MHz ¹H-NMR (DMSO-d₆, ppm) δ 10.95 (1H, s) 8.75-8.71 (1H, m) 8.57 (1H, dd, J=4.8, 1.9 Hz) 8.15 (1H, dd, J=7.6, 1.9 Hz) 7.99-7.92 (1H, m) 7.86-7.78 (1H, m) 7.70 (1H, d, J=8.4 Hz) 7.66-7.56 (3H, m) 7.26 (1H, dd, J=8.4, 1.4 Hz) 2.45 (3H, s).

### Example 11

### N-[3-(5-tert-Butylbenzoxazol-2-yl)phenyl]-3,5-dichlorobenzamide

(a) 5-tert-Butyl-2-(3-nitrophenyl)benzoxazole

The sub-title compound was prepared in accordance with Example 1, step (a) from 2-amino-4-tert-butylphenol and 3-nitrobenzoyl chloride.

(b) 3-(5-tert-Butylbenzoxazol-2-yl)phenylaniine

The sub-title compound was prepared in accordance with Example 1, step (b) from 5-tert-butyl-2-(3-nitrophenyl)benzoxazole (see step (a) above).

(c) N-[3-(5-tert-Butylbenzoxazol-2-yl)phenyl]-3,5-dichlorobenzamide

The title compound was prepared in accordance with Example 1, step (c) from 3-(5-tert-butylbenzoxazol-2-yl)phenylamine (see step (b) above) and 3,5-dichlorobenzoyl chloride.

200 MHz ¹H-NMR (DMSO-d₆, ppm) δ 10.7 (1H, s) 8.71 (1H, dd, J=1.6, 1.6 Hz) 8.03-7.87 (5H, m) 7.79-7.77 (1H, m) 7.69 (1H, d, J=8.4 Hz) 7.59 (1H, dd, J=8.0, 8.0 Hz) 7.49 (1H, dd, J=8.8, 1.8 Hz) 1.35 (9H, s).

### Example 12

### 3.5-Dichloro-N-[3-(5-ethanesulfonylbenzoxazol-2-yl)phenyl]benzamide

(a) 5-Ethanesulfonyl-2-(3-nitrophenyl)benzoxazole

The sub-title compound was prepared in accordance with Example 1, step (a) from 2-amino-4-ethanesulfonylphenol and 3-nitrobenzoyl chloride.

(b) 3-(5-Ethylsulfonylbenzoxazol-2-yl)phenylamine

The sub-title compound was prepared in accordance with Example 1, step (b) from 5-ethanesulfonyl-2-(3-nitrophenyl)benzoxazole (see step (a) above).

(c) 3.5-Dichloro-N-[3-(5-ethanesulfonylbenzoxazol-2-yl)phenyl]benzamide

The title compound was prepared in accordance with Example 1, step (c) from 3-(5-ethanesulfonylbenzoxazol-2-yl)phenylamine (see step (b) above) and 3,5-dichlorobenzoyl chloride.

200 MHz ¹H-NMR (DMSO-d₆, ppm) δ 10.7 (1H, s) 8.77 (1H, dd, J=1.6, 1.6 Hz) 8.32 (1H, d, J=1.6 Hz) 8.12-7.97 (6H, m) 7.94-7.89 (1H, m) 7.64 (1H, dd, J=8.0, 8.0 Hz) 3.38 (2H, q, J=7.4 Hz) 1.12 (3H, t, J=7.4 Hz).

### Example 13

### 3.5-Dichloro-N-[3-(5-chlorobenzoxazol-2-yl)phenyl]benzamide

(a) 5-Chloro-2-(3-nitrophenyl)benzoxazole

The sub-title compound was prepared in accordance with Example 1, step (a) from 2-amino-4-chlorophenol and 3-nitrobenzoyl chloride.

(b) 3-(5-Chlorobenzoxazol-2-yl)phenylamine

The sub-title compound was prepared in accordance with Example 1, step (b) from 5-chloro-2-(3-nitrophenyl)benzoxazole (see step (a) above).

(c) 3.5-Dichloro-N-[3-(5-chlorobenzoxazol-2-yl)phenyl]benzamide

The title compound was prepared in accordance with Example 1, step (c) from 3-(5-chlorobenzoxazol-2-yl)phenylamine (see step (b) above) and 3,5-dichlorobenzoyl chloride.

200 MHz ¹H-NMR (DMSO-d₆, ppm) δ 10.6 (1H, s) 8.71 (1H, dd, J=1.6, 1.6 Hz) 8.02-7.85 (6H, m) 7.83 (1H, d, J=8.8 Hz) 7.59 (1H, dd, J=8.0, 8.0 Hz) 7.49 (1H, dd, J=8.8, 2.0 Hz).

### Example 14

### 3.5-Dichloro-N-{3-[6-(1,1-dimethylpropyl)benzoxazol-2-yl]phenyl}benzamide

(a) 6(1,1-Dimethylpropyl)-2-(3-nitrophenyl)benzoxazole

The sub-title compound was prepared in accordance with Example 1, step (a) from 2-amino-5-(1,1-dimethylpropyl)phenol and 3-nitrobenzoyl chloride.

(b) 3-[6-(1,1-Dimethylpropyl)benzoxazol-2yl]phenylamine

The sub-title compound was prepared in accordance with Example 1, step (b) from 6-(1,1-dimethylpropyl)-2-(3-nitrophenyl)benzoxazole (see step (a) above).

(c) 3.5-Dichloro-N-{3-[6-(1,1-dimethylpropyl)benzoxazol-2yl]phenyl}benzamide

The title compound was prepared in accordance with Example 1, step (c) from 3-[6-(1,1-dimethylpropyl)benzoxazol-2-yl]phenylamine (see step (b) above) and 3,5-dichlorobenzoyl chloride.

200 MHz ¹-NMR (DMSO-d₆, ppm) δ 10.64 (1H, s) 8.70 (1H, dd, J=1.6, 1.6 Hz) 8.02-7.96 (3H, m) 7.94-7.89 (1H, m) 7.85 (1H, dd, J=2.0, 2.0 Hz) 7.72-7.71 (1H, m) 7.67 (1H, d, J=8.8 Hz) 7.58 (1H, dd, J=8.0, 8.0 Hz) 7.40 (1H, dd, J=8.8, 1.8 Hz) 1.66 (2H, q, J=7.4 Hz) 1.30 (6H, s) 0.61 (3H, t, J=7.4 Hz).

### Example 15

### 2-Chloro-N-(4-chloro-3-(5-chlorobenzoxazol-2-yl)phenyl)-5-nitrobenzamide

(a) 4-Chloro-3-(5-chlorobenzoxazol-2-yl)phenylamine

The sub-title compound was prepared in accordance with Example 1, steps (a) and

(b) from 2-amino-4-chlorophenol and 2-chloro-5-nitrobenzoyl chloride, followed by reduction of the nitro group.

(b) 2-Chloro-N-(4-chloro-3-(5chlorobenzoxazol-2-yl)phenyl)-5-nitrobenzamide

The title compound was prepared in accordance with Example 1, step (c) from 4-chloro-3-(5-chlorobenzoxazol-2-yl)phenylamine (see step (a) above) and 2-chloro-5-nitrobenzoyl chloride.

600 MHz ¹H-NMR (DMSO-d₆, ppm) δ 11.10 (1H, s) 8.66 (1H, d, J=2.6 Hz) 8.54 (1H, d, J=2.8 Hz) 8.34 (1H, dd, J=8.8, 2.8 Hz) 8.00 (1H, d, J=2.1 Hz) 7.90 (1H, d, J=8.8 Hz) 7.88 (1H, d, J=8.8 Hz) 7.86 (1H, dd, J=8.8, 2.6 Hz) 7.72 (1H, d, J=8.8Hz) 7.52 (1H, dd, J=8.8, 2.1 Hz).

### Example 16

### N-(4-Chloro-3-(5methylbenzoxazol-2-yl)phenyl)pyrazine-2-carboxamide

(a) 2-(2-Chloro-5-nitrophenyl)-5-methylbenzoxazole

The sub-title compound was prepared in accordance with Example 1, step (a) from 2-amino-4-methylphenol and 2-chloro-5nitrobenzoyl chloride.

(b) 4-Chloro-3-(5-methylbenzoxazol-2-yl)phenylamine

To a stirred suspension of 2-(2-chloro-5-nitrophenyl)-5-methylbenzoxazole (3.27 g, 11.35 mmol; see step (a) above) in EtOH (60 mL) was added NH₄Cl (aq, sat, 25mL) and Fe powder (3.62 g, 64.9 mmol). After heating at reflux for 30 min; the mixture was filtered through Celite®. EtOAc (300 mL) was added and the mixture was washed with NaHCO₃ (aq, sat) and NaCl (aq, sat) and dried (Na₂SO₄). Concentration and purification by chromatography afforded the title compound (2.14g mg, 73%)

(c) N-(4-Chloro-3-(5-methylbenzoxazol-2-yl)phenyl)pyrazine-2-carboxamide

The title compound was prepared in accordance with Example 1, step (c) from 4-chloro-3-(5-methylbenzoxazol-2-yl)phenylamine (see step (b) above) and pyrazine-2-carbonyl chloride.

200 MHz ¹H-NMR (DMSO-d₆, ppm) δ 11.17 (1H, s) 9.31 (1H, d, J=1.4 Hz) 8.94 (1H, d, J=2.6 Hz) 8.88 (1H, d, J=2.6 Hz) 8.82 (1H, dd, J=2.4, 1.6 Hz) 8.10 (1H, dd, J=8.8, 2.6 Hz) 7.72-7.67 (3H, m) 7.32-7.27 (1H, m) 2.45 (3H, s).

### Example 17

### N-[4-Chloro-3-(5-methylbenzoxazol-2-yl)phenyl]-2-trifluoromethylbenzamide

The title compound was prepared in accordance with Example 1, step (c) from 4-chloro-3-(5-methylbenzoxazol-2-yl)phenylamine (see Example 16, step (b)) and 2-trifluoromethylbenzoyl chloride.

200 MHz ¹H-NMR (DMSO-d₆, ppm) δ 10.96 (1H, s) 8.64 (1H, d, J=2.4 Hz) 7.88-7.65 (8H, m) 7.28 (1H, dd, J=8.4, 1.2 Hz) 2.44 (3H, s).

### Example 18

### N-[4-Chloro-3-(5-methylbenzoxazol-2-yl)phenyl]-2-nitrobenzamide

The title compound was prepared in accordance with Example 1, step (c) from 4-chloro-3-(5-methylbenzoxazol-2-yl)phenylaniine (see Example 16, step (b)) and 2-nitrobenzoyl chloride.

200 MHz ¹H-NMR (DMSO-d₆, ppm) δ 11.05 (1H, s) 8.60 (1H, d, J=2.4 Hz) 8.17 (1H, d, J=7.8 Hz) 7.93-7.67 (7H, m) 7.29 (1H, dd, J=8.4, 1.2 Hz) 2.44 (3H, s).

### Example 19

### 2-Chloro-N-[4-chloro-3-(5-methylbenzoxazol-2-yl)phenyl]nicotinamide

The title compound was prepared in accordance with Example 1, step (c) from 4-chloro-3-(5-methylbenzoxazol-2-yl)phenylamine (see Example 16, step (b)) and 2-chloronicotinoyl chloride.

200 MHz ¹H-NMR (DMSO-d₆, ppm) δ 11.05 (1H, s) 8.65 (1H, d, J=2.5 Hz) 8.57 (1H, dd, J=4.8, 1.9 Hz) 8.16 (1H, dd, J=7.6, 1.9 Hz) 7.87 (1H, dd, J=8.8, 2.6 Hz) 7.75-7.67 (3H, m) 7.60 (1H, dd, J=7.6, 4.8 Hz) 7.30 (1H, dd, J=8.2, 1.4 Hz) 2.47 (3H, s).

### Example 20

### N-[4-Chloro-3-(5-methylbenzoxazol-2-yl)phenyl]-2-trifluoromethoxybenzamide

The title compound was prepared in accordance with Example 1, step (c) from 4-chloro-3-(5-methylbenzoxazol-2-yl)phenylainine (see Example 16, step (b)) and 2-trifluoromethoxybenzoyl chloride.

200 MHz ¹H-NMR (DMSO-d₆, ppm) δ 10.89 (1H, s) 8.64 (1H, d, J=2.4 Hz) 7.87 (1H, dd, J=8.8, 2.6 Hz) 7.78-7.63 (5H, m) 7.58-7.49 (2H, m) 7.28 (1H, dd, J=8.4 1.4 Hz)2.44 (3H, s).

### Example 21

### N-[4-Chloro-3-(5-methylbenzoxazol-2-yl)phenyl]-2-toluamide

The title compound was prepared in accordance with Example 1, step (c) from 4-chloro-3-(5-methylbenzoxazol-2-yl)phenylamine (see Example 16, step (b)) and 2-toluoyl chloride.

200 MHz ¹H-NMR (DMSO-d₆, ppm) δ 10.67 (1H, s) 8.70 (1H, d, J=2.6 Hz) 7.89 (1H, dd, J=8.8, 2.6 Hz) 7.72-7.64 (3H, m) 7.53-7.26 (5H, m) 2.44 (3H, s) 2.39 (3H, s).

### Example 22

### N-[4-Chloro-3-(5methylbenzoxazol-2-yl)phenyl]-2-anisoylamide

The title compound was prepared in accordance with Example 1, step (c) from 4-chloro-3-(5-methylbenzoxazol-2-yl)phenylamine (see Example 16, step (b)) and 2-anisoyl chloride.

200 MHz ¹H-NMR (DMSO-d₆, ppm) δ 10.47 (1H, s) 8.67 (1H, d, J=2.6 Hz) 7.90 (1H, dd, J=8.8, 2.6 Hz) 7.72-7.59 (4H, m) 7.55-7.46 (1H, m) 7.28 (1H, dd, J=8.6, 1.6 Hz) 7.17 (1H, d, J=8.4 Hz) 7.06 (1H, ddd, J=7.4, 7.4, 0.8 Hz) 3.88 (3H, s) 2.45 (3H, s).

### Example 23

### N-[4-Chloro-3-(5-methylbenzoxazol-2-yl)phenyl]-2-fluorobenzamide

The title compound was prepared in accordance with Example 1, step (c) from 4-chloro-3-(5-methylbenzoxazol-2-yl)phenylamine (see Example 16, step (b)) and 2-fluorobenzoyl chloride.

200 MHz ¹H-NMR (DMSO-d₆, ppm) δ 10.80 (1H, s) 8.65 (1H, d, J=2.6 Hz) 7.90 (1H, dd, J=8.8, 2.6 Hz) 7.74-7.54 (5H, m) 7.41-7.26 (3H, m) 2.44 (3H, s).

### Example 24

### 2-Chloro-N-[4-chloro-3-(5-methylbenzoxazol-2yl)phenyl]benzamide

The title compound was prepared in accordance with Example 1, step (c) from 4-chloro-3-(5-methylbenzoxazol-2-yl)phenylamine (see Example 16, step (b)) and 2-chlorobenzoyl chloride.

200 MHz ¹H-NMR (DMSO-d₆, ppm) δ 10.94 (1H, s) 8.70 (1H, d, J=2.4 Hz) 7.90 (1H, dd, J=8.8, 2.4 Hz) 7.76-7.46 (7H, m) 7.32 (1H, dd, J=8.6, 1.6 Hz) 2.48 (3H, s).

### Example 25

### 2-Chloro-N-[4-methyl-3-(5-methylbenzoxazol-2yl)phenyl]nicotinamide

(a) 4-Methyl-3-(5-methylbenzoxazol-2-yl)phenylamine

The sub-title compound was prepared in accordance with Example 1, steps (a) and (b) from 2-amino-4-methylphenol and 2-methyl-5-nitrobenzoyl chloride, followed by reduction of nitro group.

(b) 2-Chloro-N-[4-methyl-3-(5-methylbenzoxazol-2-yl)phenyl]nicotinamide

The title compound was prepared in accordance with Example 1, step (c) from 4-methyl-3-(5-methylbenzoxazol-2-yl)phenylamine (see step (a) above) and 2-chloronicotinoyl chloride.

200 MHz ¹H-NMR (DMSO-d₆, ppm) δ 10.85 (1H, s) 8.61 (1H, d, J=2.2 Hz) 8.56 (1H, dd, J=4.8, 1.8 Hz) 8.14 (1H, dd, J=7.6, 1.8 Hz) 7.75 (1H, dd, J=8.4, 2.2 Hz) 7.69 (1H, d, J=8.4 Hz) 7.66-7.63 (1H, m) 7.59 (1H, dd, J=7.6, 4.8 Hz) 7.45 (1H, d, J=8.4 Hz) 7.27 (1H, dd, J=8.4, 1.2 Hz) 2.73 (3H, s) 2.46 (3H, s).

### Example 26

### 4-Amino-N-[3-(5-methylbenzoxazol-2-yl)phenyl]benzamide

(a) 2-(3-Bromophenyl)-5-methylbenzoxazole

The sub-title compound was prepared in accordance with Example 1, step (a) from 2-amino-4-methylphenol and 3-bromobenzoyl chloride.

(b) 4-Amino-N-[3-(5-methylbenzoxazol-2-yl)phenyl]benzamide

A mixture of 2-(3-bromophenyl)-5-methylbenzoxazole (144 mg, 0.50 mmol; see step (a) above), CuI (12 mg, 0.06 mmol), K₃PO₄ (254 mg, 1.2 mmol), N,N′-dimethyl-1,2-diaminoethane (20 μL, 0.18 mmol), 4-aminobenzamide (68.1 mg, 0.5 mmol) and toluene (2 mL) was heated at 110° C. for 48 h. The mixture was diluted with EtOAc (70 mL), filtered through Celite®, dried (Na₂SO₄) and concentrated. The residue was recrystallised from DMF to afford the title compound (110 mg, 65%).

200 MHz ¹H-NMR (DMSO-d₆, ppm) δ 10.02 (1H, s) 8.75-8.70 (1H, m) 8.02-7.94 (1H, m) 7.87-7.80 (1H, m) 7.80-7.82 (2H, m) 7.65 (1H, d, J=8.4 Hz) 7.61-7.57 (1H, m) 7.57-7.46 (1H, m) 7.23 (1H, dd, J=8.4, 1.1 Hz) 6.65-6.56 (2H, m) 5.80 (2H, s) 2.43 (3H,s).

### Example 27

### 3-Amino-4-methyl-N-[3-(5-methylbenzoxazol-2-yl)phenyl]benzamide

The title compound was prepared in accordance with Example 26, step (b) from 2-(3-bromophenyl)-5-methylbenzoxazole (see Example 26, step (a)) and 3-amino-4-methylbenzamide.

200 MHz ¹H-NMR (DMSO-d₆, ppm) δ 10.27 (1H, s) 8.75-8.71 (1H, m) 8.01-7.94 (1H, m) 7.90-7.83 (1H, m) 7.66 (1H, d, J=8.4 Hz) 7.62-7.58 (1H, m) 7.54 (1H, dd, J=8.1, 8.1 Hz) 7.27-7.17 (2H, m) 7.12 (1H, dd, J=7.7,1.8 Hz) 7.05 (1H, d, J=7.7 Hz) 5.09 (2H, s) 2.43 (3H, s) 2.11 (3H, s).

### Example 28

### N-[4-Isopropoxy-3-(5-methylbenzoxazol-2-yl)phenyl]-2-trifluoromethoxybenzamide

(a) 4-Bromo-2-(5-methylbenzoxazol-2yl)phenol

A mixture of 2-amino-4-methylphenol (18 mmol, 2.22 g) and 5-bromo-2-hydroxybenzoyl chloride (20 mmol, 4.69 g) in 25 mL of 1,4-dioxane was placed in 10 microwave process vials and each of the sealed reaction vessels was treated with microwaves for 15 min at 210° C. After cooling, the reaction mixture was filtered through Celite®. The filter cake was washed with EtOAc. The combined filtrates were concentrated and purified by chromatography to give the sub-title compound (3.91 g, 72%).

(b) 2-(5-Bromo-2-isopropoxyphenyl)-5-methylbenzoxazole

The sub-title compound was prepared from 4-bromo-2-(5-methylbenzoxazol-2-yl)-phenol (see step (a) above) and 2-bromopropane in accordance with the following general procedure. For example, a solution of 4-bromo-2-(5-methylbenzoxazol-2-yl)phenol (see step (a) above) in dry DMF may be added gradually to a suspension of 75% NaH (washed twice with dry Et₂O prior to use) in DMF at 0° C. The reaction mixture may then be stirred at 0° C. for e.g. 30 min, whereupon 2-bromopropane in DMF may be added. After stirring at room temperature for e.g. 24 h, the mixture may then be poured into water and extracted (e.g. with MeOtBu). The combined extracts may then be washed with water and brine and then dried over Na₂SO₄. Concentration under reduced pressure and purification by chromatography may then afford the sub-title compound.

(c) 2(5-Iodo-2-isopropoxyphenyl)-5-methylbenzoxazole

The sub-title compound was prepared from 2-(5-bromo-2-isopropoxyphenyl)-5-methylbenzoxazole (see step (b) above) in accordance with the following general procedure. For example, an oven dried ACE® pressure tube may be charged with 2-(5-bromo-2-isopropoxyphenyl)-5-methylbenzoxazole (see step (b) above), CuI and NaI. The reaction tube may then be purged with argon, and 1,4-dioxane may then be added followed by N,N′-dimethyl-1,2-diaminoethane. The reaction mixture may then be heated at 130° C. for 18 h. The mixture was filtered through Celite®. Solvent removal under reduced pressure and chromatography afforded the sub-title compound (702 mg, 76%).

(d) N-[4-Isopropoxy-3-(5-methylbenzoxazol-2-yl)phenyl]-2-trifluoromethoxybenzamide

The title compound was prepared from 2-(5-iodo-2-isopropoxyphenyl)-5-methylbenzoxazole (see step (c) above) and 2-(trifluoromethoxy)benzamide in accordance with the following general procedure. For example, a mixture of 2-(5-iodo-2-isopropoxyphenyl)-5-methylbenzoxazole, CuI, K₃PO₄, N,N′-dimethyl-1,2-diaminoethane, 2-(trifluoromethoxy)benzamide and toluene may be heated at 110° C. for 48 h. The mixture may then be diluted with EtOAc, filtered through Celite®, dried (Na₂SO₄) and concentrated. The residue may then be recrystallised from DMF to afford the title compound.

200 MHz ¹H-NMR (CDCl₃, ppm) δ 8.27 (1H, s) 8.16 (1H, d, J=2.7 Hz) 8.10 (1H, dd, J=7.6, 1.9 Hz) 7.94 (1H, dd, J=9.0, 2.7 Hz) 7.63-7.52 (2H, m) 7.51-7.41 (2H, m) 7.40-7.33 (1H, m) 7.20-7.09 (2H, m) 4.61 (1H, septet, J=6.0 Hz) 2.49 (3H, s) 1.42 (6H, d, J=6.0 Hz).

### Example 29

### 2-Amino-5-chloro-N-[4-methoxy-3-(5-methylbenzoxazol-2-yl)-phenyl]benzamide

The title compound was prepared from 2-(5-iodo-2-methoxyphenyl)-5-methylbenzoxazole (see Example 28 step (c)) and 2-amino-5-chlorobenzamide in accordance with Example 28, step (d).

200 MHz ¹H-NMR (CDCl₃, ppm) δ 8.21 (1H, d, J=2.7 Hz) 7.84 (1H, dd, J=9.1, 2.7 Hz) 7.80 (1H, s) 7.60-7.56 (1H, m) 7.50-7.43 (2H, m) 7.21 (1H, dd, J=8.7, 2.2 Hz) 7.18-7.13 (1H, m) 7.09 (1H, d, J=9.1 Hz) 6.67 (1H, d, J=8.7 Hz) 5.53 (2H, s) 4.03 (3H, s) 2.48 (3H, s).

### Example 30

### N-[4-Hydroxy-3-(5-methylbenzoxazol-2-yl)phenyl]-2-trifluoromethylbenzamide

(a) 2-(2-Benzyloxy-5-bromophenyl)-5-methylbenzoxazole

The sub-title compound was prepared from 4-bromo-2-(5-methylbenzoxazol-2-yl)phenol (see Example 28 step (a)) and chloromethylbenzene in accordance with Example 28 step (b).

(b) 2-(2-Benzyloxy-5-iodophenyl)-5-methylbenzoxazole

The sub-title compound was prepared from 2-(2-benzyloxy-5-bromophenyl)-5-methylbenzoxazole (see step (a) above) in accordance with Example 28 step (c).

(c) N-[4-Benzyloxy-3-(5-methylbenzoxazol-2-yl)phenyl]-2-hydroxybenzamide

The sub-title compound was prepared from 2-(2benzyloxy-5-iodophenyl)-5-methylbenzoxazole (see step (b) above) and 2-trifluoromethylbenzamide in accordance with Example 28, step (d).

(d) N-[4-Hydroxy-3-(5-methylbenzoxazol-2-yl)phenyl]-2trifluoromethylbenzamide

A solution of N-[4-benzyloxy-3-(5-methylbenzoxazol-2-yl)phenyl]-2-hydroxybenzamide (270 mg, 0.54 mmol; see step (c) above) in EtOAc (20 mL) and EtOH (10 mL) was hydrogenated in the presence of 10% Pd-C (140 mg) at room temperature for 2 hours. The mixture was filtered through Celite®. Solvent removal under reduced pressure and chromatography and recrystallization from EtOH afforded the title compound (140 mg, 63%).

200 MHz ¹H-NMR (DMSO-d₆, ppm) δ 11.14 (1H, s) 10.66 (1H, s) 8.60 (1H, d, J=2.5 Hz) 7.92-7.60 (7H, m) 7.31 (1H, dd, J=8.6, 1.2 Hz) 7.14 (1H, d, J=9.0 Hz) 2.46 (3H, s).

### Example 31

### N-[4-Isopropoxy-3-(5-methylbenzoxazol-2-yl)phenyl]-2-trifluoromethylbenzamide

The title compound was prepared from 2-(5-iodo-2-isopropoxyphenyl)-5-methylbenzoxazole (see Example 28 step (c)) and 2-trifluoromethylbenzamide in accordance with Example 28, step (d).

200 MHz ¹H-NMR (DMSO₆, ppm) δ 10.66 (1H, s) 8.47 (1H, d, J=2.6 Hz) 7.90-7.59 (7H, m) 7.29 (1H, d, J=9.2 Hz) 7.24 (1H, dd, J=8.5, 1.6 Hz) 4.68 (1H, septet, J=6.0 Hz) 2.45 (3H, s) 1.33 (6H, d, J=6.0 Hz).

### Example 32

### N-[4-Cyclopentyloxy-3-(5-methylbenzoxazol-2-yl)phenyl]-2-trifluoromethylbenzamide

(a) 2-(5-Bromo-2-cyclopentyloxyphenyl)-5-methylbenzoxazole

The sub-title compound was prepared from 4-bromo-2-(5-methylbenzoxazol-2-yl)phenol (see Example 28 step (a)) and bromocyclopentane in accordance with Example 28 step (b).

(b) 2-(2-Cyclopentyloxy-5-iodophenyl)-5-methyl-benzoxazole

The sub-title compound was prepared from 2-(5-bromo-2-cyclopentyloxyphenyl)-5-methylbenzoxazole (see step (a) above) in accordance with Example 28 step (c).

(c) N-[4-Cyclopentyloxy-3-(5-methylbenzoxazol-2-yl)phenyl]-2-trifluoromethylbenzamide

The title compound was prepared from 2-(2-cyclopentyloxy-5-iodophenyl)-5- methylbenzoxazole (see step (b) above) and 2-trifiuoromethylbenzamide in accordance with Example 28, step (d).

200 MHz ¹H-NMR (DMSO-d₆, ppm) δ 10.64 (1H, s) 8.47 (1H, d, J=2.6 Hz) 7.90-7.67 (5H, m) 7.64-7.58 (2H, m) 7.29 (1H, d, J=9.2 Hz) 7.23 (1H, dd, J=8.6, 1.5 Hz) 5.04-4.95 (1H, m) 2.45 (3H, s) 1.95-1.51 (8H, m).

### Example 33

### N-[3-(5-Bromobenzoxazol-2-yl)-4-chlorophenyl]-2,5-dichloroberizamide

(a) 5-Bromo-2-(2-chloro-5-nitrophenynbenzoxazole

The sub-title compound was prepared from 2-amino-4-bromophenol and 2-chloro-5-nitrobenzoyl chloride in accordance with Example 1 step (a).

(b) 3-(5-Bromobenzoxazol-2-yl)-4-chlorophenylamine

The sub-title compound was prepared from 5-bromo-2-(2-chloro-5-nitrophenyl)benzoxazole (see step (a) above) in accordance with Example 34 step (b) below.

(c) N-[3-(5-Bromobenzoxazol-2-yl)-4-chlorophenyl]-2,5-dichlorobenzamide

The title compound was prepared from 3-(5-bromobenzoxazol-2-yl)-4-chlorophenylamine (see step (b) above) and 2,5-dichlorobenzoyl chloride in accordance with Example 1 step (c).

200 MHz ¹H-NMR (DMSO-d₆, ppm) δ 11.01 (1H, s) 8.70 (1H, d, J=2.5 Hz) 8.17 (1H, d, J=1.9 Hz) 7.88 (1H, dd, J=8.8, 2.6 Hz) 7.86 (1H, d, J=8.8 Hz) 7.83 (1H, d, J=1.6 Hz) 7.73 (1H, d, J=8.8 Hz) 7.66 (1H, dd, J=8.6, 1.9 Hz) 7.64-7.58 (2H, m).

### Example 34

### 2-Amino-N-[4-chloro-3-(5methylbenzoxazol-2-yl)phenyl]benzamide

(a) 2-(2-Chloro-5-nitrophenyl)-5-methylbenzoxazole

The sub-title compound was prepared in accordance with Example 1, step (a) from 2-amino-4-methylphenol and 2-chloro-5-nitrobenzoyl chloride.

(b) 4-Chloro-3-(5-methylbenzoxazol-2-yl)phenylamine

To a stirred suspension of 2-(2-chloro-5-nitrophenyl)-5-methylbenzoxazole (3.27 g, 11.35 mmol; see step (a) above) in EtOH (60 mL) was added NH₄Cl (aq, sat, 25mL) and Fe powder (3.62 g, 64.9 mmol). After heating at reflux for 30 min, the mixture was filtered through Celite®. EtOAc (300 mL) was added and the mixture was washed with NaHCO₃ (aq, sat) and NaCl (aq, sat) and dried (Na₂SO₄). Concentration and purification by chromatography afforded the title compound (2.14 g mg, 73%)

(c) N-[4-Chloro-3-(5-methylbenzoxazol-2-yl)phenyl]-2-nirobenzamide

The sub-title compound was prepared in accordance with Example 1, step (c) from 4-chloro-3-(5-methylbenzoxazol-2-yl)phenylamine (see step (b) above) and 2-nitrobenzoylchloride.

(d) 2-Amino-N-[4-chloro-3-(5-methylbenzoxazol-2-yl)phenyl]benzamide

The title compound was prepared from N-[4-chloro-3-(5-methylbenzoxazol-2-yl)phenyl]-2-nitrobenzamide (see step (c) above) by reduction of the nitro group in accordance with step (b) above.

200 MHz ¹H-NMR (DMSO-d₆, ppm) δ 10.33 (1H, s) 8.67-8.64 (1H, m) 7.95 (1H, dd, J=8.8, 2.6 Hz) 7.72-7.62 (4H, m) 7.31-7.17 (2H, m) 6.75 (1H, d, J=8.4 Hz) 6.63-6.55 (1H, m) 6.41 (2H, b.s) 2.45 (3H, s).

### Example 35

### N-[4-Chloro-3-(5-methylbenzoxazol-2-yl)phenyl]benzamide

The title compound was prepared in accordance with Example 1, step (c) from 4-chloro-3-(5-methybenzoxazol-2-yl)phenylamine (see Example 34, step (b)) and benzoyl chloride.

200 MHz ¹H-NMR (DMSO-d₆, ppm) δ 10.60 (1H, s) 8.70 (1H, d, J=2.6 Hz) 8.04 (1H, dd, J=8.8, 2.6 Hz) 8.01-7.96 (2H, m) 7.72-7.65 (3H, m) 7.61-7.49 (3H, m) 7.28 (1H, dd, J=8.4, 1.6 Hz) 2.45 (3H, s).

### Example 36

### N-[4-Chloro-3-(5-methylbenzoxazol-2-yl)phenyl]-4-methoxybenzamide

The title compound was prepared in accordance with Example 1, step (c) from 4-chloro-3-(5-methylbenzoxazol-2-yl)phenylamine (see Example 34, step (b)) and 4-methoxybenzoyl chloride.

200 MHz ¹H-NMR (DMSO-d₆, ppm) δ 10.43 (1H, s) 8.68 (1H, d, J=2.6 Hz) 8.06-7.95 (3H, m) 7.71-7.63 (3H, m) 7.31-7.26 (1H, m) 7.10-7.03 (2H, m) 3.83 (3H, s) 2.45 (3H, s).

### Example 37

### 4-Chloro-N-[4-chloro-3-(5-methylbenzoxazol-2-yl)phenyl]Benzamide

The title compound was prepared in accordance with Example 1, step (c) from 4-chloro-3-(5-methyl-benzoxazol-2-yl)phenylamine (see Example 34, step (b)) and 4-chlorobenzoyl chloride.

200 MHz ¹H-NMR (DMSO-d₆, ppm) δ 10.64 (1H, s) 8.67 (1H, dd, J=2.6 Hz) 8.05-7.98 (3H, m) 7.71-7.58 (5H, m) 7.28 (1H, dd, J=8.4, 1.6 Hz) 2.44 (3H, s).

### Example 38

### N-[4-Chloro-3-(5-methylbenzoxazol-2-yl)phenyl]-4-methylbenzamide

The title compound was prepared in accordance with Example 1, step (c) from 4-chloro-3-(5-methylbenzoxazol-2-yl)phenylamine (see Example 34, step (b)) and 4-methylbenzoyl chloride.

200 MHz ¹ H-NMR (DMSO-d₆, ppm) δ 10.5 (1H, s) 8.69 (1H, d, J=2.6 Hz) 8.04 (1H, dd, J=8.8, 2.6 Hz) 7.94-7.88 (2H, m) 7.71-7.63 (3H, m) 7.36-7.26 (3H, m) 2.44 (3H, s) 2.37 (3H, s).

### Example 39

### 3.4-Dichloro-N-[4-chloro-3-(5-methylbenzoxazol-2-yl)phenyl]benzamide

The title compound was prepared in accordance with Example 1, step (c) from 4-chloro-3-(5-methylbenzoxazol-2-yl)phenylamine (see Example 34, step (b)) and 3,4-dichlorobenzoyl chloride.

200 MHz 1H-NMR (DMSO-d₆, ppm) δ 10.7 (1H, s) 8.64 (1H, d, J=2.6 Hz) 8.25 (1H, d, J=2.0 Hz) 8.03 (1H, dd, J=8.8, 2.6 Hz) 7.96 (1H, dd, J=8.4, 2.0 Hz) 7.82 (1H, d, J=8.4 Hz) 7.71-7.65 (3H, m) 7.28 (1H, dd, J=8.4, 1.4 Hz) 2.44 (3H, s).

### Example 40

### N-[4-Dimethylamino-3-(5-methylbenzoxazol-2-yl)-phenyl]-2-trifluoromethylbenzamide

(a) Dimethyl-[2-(5-methylbenzoxazol-2-yl)-4-nitrophenyl]amine

An oven dried ACE® pressure tube was charged with 2-(2-chloro-5-nitrophenyl)-5-methylbenzoxazole (790 mg, 2.74 mmol; see Example 34, step (a)), CuCl (49 mg, 0.49 mmol) and copper powder (47 mg, 0.74 mmol). Liquid N,N-dimethylamine (15 mL) was added and the reaction mixture was heated at 60° C. for 48 h. After cooling to −40° C. the pressure tube was opened, liquid N,N-dimethylamine was allowed to evaporate and the residue was dissolved in CH₂Cl₂. Filtration of inorganic material and solvent removal under reduced pressure afforded the crude sub-title compound (897 mg), which was used in the subsequent step without further purification.

(b) N′,N′-Dimethyl-2-(5-methylbenzoxazol-2-yl)-benzene-1,4-diamine hydrochloride

A solution of dimethyl-[2-(5-methylbenzoxazol-2-yl)-4-nitrophenyl]amine (897 mg, 3 mmol; see step (a) above) in glacial AcOH (50 mL) was stirred at ambient temperature under 4 atm H₂ pressure in the presence of 10% Pd on carbon (344 mg; 3.23 mmol) for 2.5 h. After filtration through Celite® the solvent was evaporated, the residue dissolved in EtOAc (100 mL) and washed with aq. saturated NaHCO₃. After drying and solvent removal under reduced pressure, the residue was dissolved in dry diethylether and the product was precipitated as a hydrochloric acid salt after treatment with gaseous HCl to afford 500 mg (55%) of the sub-title compound.

(c) N-[4-Dimethylamino-3-(5-methylbenzoxazol-2yl)phenyl]-2-trifluoromethylbenzamide

A mixture of N′,N′-dimethyl-2-(5-methylbenzoxazol-2-yl)-benzene-1,4-diamine hydrochloride (250 mg, 0.82 mmol; see step (b) above), 2-trifluoromethylbenzoyl chloride (232 mg, 1.11 mmol) and triethylamine (215 μL, 2.96 mmol) in dry THF (20 mL) was heated under reflux for 24 h. Evaporation of solvent and purification by chromatography, followed by recrystallization from EtOAc-hexanes, afforded the title compound (56 mg, 14%).

200 MHz ¹H-NMR (DMSO-d₆, ppm) δ 10.6 (1H, s) 827 (1H, d, J=2.5 Hz) 7.86-7.59 (7H, m) 7.23-7.18 (1H, m) 7.14 (1H, d, J=9.0 Hz) 2.66 (6H, s) 2.43 (3H, s).

### Example 41

### N-[3-(5-Methylbenzoxazol-2-yl)-4-pyrrolidin-1-yl-phenyl]-2-trifluoromethylbenzamide

(a) 5-Methyl-2-(5-nitro-2-pyrrolidin-1-yl-phenyl)benzoxazole

The sub-title compound was prepared in accordance with Example 40, step (a) from 4-chloro-3-(5-methylbenzoxazol-2-yl)phenylamine (Example 34, step (b)) and pyrrolidine.

(b) 3-(5-Methylbenzoxazol-2-yl)-4-pyrrolidin-1-yl-phenylamine hydrochloride

The sub-title compound was prepared in accordance with Example 40, step (b) from 5-methyl-2-(5-nitro-2-pyrrolidin-1-yl-phenyl)benzoxazole (see step (a) above).

(c) N-[3-(5-Methylbenzoxazol-2-yl)-4-pyrrolidin-1-yl-phenyl]2-trifluoromethylbenzamide

The title compound was prepared in accordance with Example 40, step (c) from 3-(5-menthybenzoxazol-2-yl)-4-pyrrolidin-1-yl-phenylamine hydrochloride (see step (b) above) and 2-trifluoromethylbenzoyl chloride.

200 MHz ¹H-NMR (DMSO-d₆, ppm) δ 10.4 (1H, s) 8.04 (1H, d, J=2.6 Hz) 7.85-7.56 (7H, m) 7.20 (1H, dd, J=8.4, 1.4 Hz) 6.94 (1H, d, J=9.0 Hz) 3.06-2.99 (4H, m) 2.43 (3H, s) 1.82-1.76 (4H, m).

### Example 42

### N-[3-(5-tert-Butylbenzoxazol-2-yl)-4-chlorophenyl]-2-trifluoromethylbenzamide

(a) 5-tert-Butyl-2-(2-chloro-5-nitrophenyl)benzoxazole

The sub-title compound was prepared from 2-amino-4-tert-butylphenol and 2-chloro-5-nitrobenzoyl chloride in accordance with Example 1 step (a).

(b) 3-(5-tert-Butylbenzoxazol-2-yl)-4-chloro-phenylamine

The sub-title compound was prepared from 5-tert-butyl-2-(2-chloro-5-nitrophenyl)benzoxazole (see step (a) above) in accordance with Example 34 step (b).

(c) N-[3-(5-tert-Butyl-benzoxazol-2-yl)-4-chlorophenyl]-2-trifluoromethylbenzamide

The title compound was prepared from 3-(5-tert-butylbenzoxazol-2-yl)-4-chlorophenylamine (see step (b) above) and 2-trifluoromethylbenzoyl chloride in accordance with Example 1 step (c).

200 MHz ¹H-NMR (DMSO-d₆, ppm) δ 11.0 (1H, s) 8.67 (1H, d, J=2.4 Hz) 7.88-7.66 (8H, m) 7.56-7.51 (1H, m) 1.35 (9H, s).

### Example 43

### N-[4-Chloro-3-(4-methylbenzoxazol-2-yl)phenyl]-2-trifluormethylbenzamide

(a) 2-(2-Chloro-5-nitrophenyl)-4-methylbenzoxazole

The sub-title compound was prepared from 2-amino-3-methylphenol and 2-chloro-5-nitrobenzoyl chloride in accordance with Example 1 step (a).

(b) 4-Chloro-3-(4-methylbenzoxazol-2-yl)phenylamine

The sub-title compound was prepared from 2-(2-chloro-5-nitrophenyl)-4-methylbenzoxazole (see step (a) above) in accordance with Example 34 step (b).

(c) N-[4-Chloro-3-(4-methylbenzoxazol-2-yl)phenyl]-2-trifluormethylbenzamide

The title compound was prepared from 4-chloro-3-(4-methylbenzoxazol-2-yl)-phenylamine (see step (b) above) and 2-trifluoromethylbenzoyl chloride in accordance with Example 1 step (c).

200 MHz ¹H-NMR (DMSO-d₆, ppm) δ 10.98 (1H, s) 8.60 (1H, d, J=2.6 Hz) 7.92-7.58 (7H, m) 7.39-7.32 (1H, m) 728-7.22 (1H, m) 2.59 (3H, s).

### Example 44

### N-[4-Chloro-3-(5-chlorobenzoxazol-2-yl)phenyl]-2-trifluoromethylbenzamide

(a) 5-Chloro-2-(2-chloro-5-nitrophenyl)benzoxazole

The sub-title compound was prepared from 2-amino-4-chlorophenol and 2-chloro-5-nitrobenzoyl chloride in accordance with Example 1 step (a).

(b) 4-Chloro-3-(5-chlorobenzoxazol-2-yl)phenylamine

The sub-title compound was prepared from 5-chloro-2-(2-chloro-5-nitrophenyl)benzoxazole (see step (a) above) in accordance with Example 34 step (b).

(c) N-[4-Chloro-3-(5-chlorobenzoxazol-2-yl)phenyl]-2-trifluoromethylbenzamide

The title compound was prepared from 4-chloro-3-(5-chlorobenzoxazol-2-yl)-phenylamine (see step (b) above) and 2-trifluoromethylbenzoyl chloride in accordance with Example 1 step (c).

200 MHz ¹H-NMR (DMSO-d₆, ppm) δ 10.99 (1H, s) 8.69 (1H, d, J=2.6 Hz) 8.01 (1H, d, J=1.8 Hz) 7.93-7.66 (7H, m) 7.52 (1H, dd, J=8.8, 2.2 Hz).

### Example 45

### N-[4-Chloro-3-(6-chlorobenzoxazol-2-yl)phenyl]-2-trifluoromethylbenzamide

(a) 6-Chloro-2-(2-chloro-5-nitrophenyl)benzoxazole

The sub-title compound was prepared from 2-amino-5-chlorophenol and 2-chloro-5-nitrobenzoyl chloride in accordance with Example 1 step (a).

(b) 4-Chloro-3-(6-chlorobenzoxazol-2-yl)phenylamine

The sub-title compound was prepared from 6-chloro-2-(2-chloro-5-nitrophenyl)benzoxazole (see step (a) above) in accordance with Example 34 step (b).

(c) N-[4-Chloro-3-(6-chlorobenzoxazol-2-yl)phenyl]-2-trifluoromethylbenzamide

The title compound was prepared from 4-chloro-3-(6-chlorobenzoxazol-2-yl)phenylamine (see step (b) above) and 2-trifiuoromethylbenzoyl chloride in accordance with Example 1 step (c).

200 MHz ¹H-NMR (DMSO-d₆, ppm) δ 10.99 (1H, s) 8.66 (1H, d, J=2.6 Hz) 8.07 (1H, d, J=2.0 Hz) 7.93-7.67 (7H, m) 7.50 (1H, dd, J=8.6, 2.0 Hz).

### Example 46

### N-[4-Fluoro-3-(5-methylbenzoxazol-2-yl)phenyl]-2-trifluoromethoxybenzamide

(a) 2-(5-Bromo-2-fluorophenyl)-5-methylbenzoxazole

The sub-title compound was prepared from 2-amino-4-methylphenol and 5-bromo-2-fluorobenzoyl chloride in accordance with Example 28 step (a).

(b) N-[4-Fluoro-3-(5-methylbenzoxazol-2-yl)phenyl]-2-trifluoromethoxvbenzamide

The title compound was prepared from 2-(5-bromo-2-fluorophenyl)-5-methylbenzoxazole (see step (a) above) and 2-trifluoromethoxybenzamide in accordance with Example 28 step (d).

200 MHz ¹H-NMR (DMSO-d₆, ppm) δ 10.71 (1H, s) 8.71 (1H, dd, J=6.5, 2.6Hz) 7.90-7.81 (1H, m) 7.79-7.62 (4H, m) 7.59-7.22 (3H, m) 7.27 (1H, dd, J=8.5, 1.2 Hz)2.44 (3H, s).

### Example 47

### N-[4-Chloro-3-(4-chlorobenzoxazol-2-yl)phenyl]-2-trifluoromethylenzamide

(a) N-(2-Methoxyphenyl)-2,2-dimethylpropionamide

To a cooled (0° C.) solution of 2-methoxyphenylamine (7 g, 54 mmol) in CH₂Cl₂ (70 mL) was added triethylamine (10 mL, 71 mmol) and 2,2-dimethylpropionyl chloride (8.8 mL, 71 mmol). The reaction was stirred at ambient temperature for 2hours, poured into water (200 mL) and extracted with EtOAc. The combined organic extracts were washed with aqueous 1 M HCl and then aqueous saturated NaHCO₃. Concentration under reduced pressure and recrystallization from EtOAc-petroleum ether afforded the sub-title compound (15 g, 89%).

(b) N-(2-Chloro-6-methoxyphenyl)-2,2-dimethylpropionamide

To a cooled (−15° C.) solution of N-(2-methoxyphenyl)-2,2-dimethylpropionamide (5.1 g, 24.6 mmol; see step (a) above) in Et₂O under argon atmosphere was added TMEDA (3.7 mL, 24.6 mmol), followed by a 2.5 M solution of n-BuLi in hexanes (9.8 mL, 24.6 mmol). After stirring for 2 hours at −15° C., the reaction was cooled to −30° C. and a solution of C₂Cl₆ (8.15 g, 34.4 mmol) in Et₂O (30 mL) was added. The mixture was allowed to warm to ambient temperature, poured into 1 M HCl (100 mL) and extracted with EtOAc. Concentration and purification by chromatography afforded the sub-title compound (2.44 g, 41%).

(c) 2-Chloro-6-methoxyphenylamine

A mixture of N-(2-chloro-6-methoxyphenyl)-2,2-dimethylpropionamide (3.89 g, 16.1 mmol; see step (b) above), AcOH (40 mL) and aqueous concentrated HCl (20mL) was heated at 75° C. for 72 hours, and then cooled and neutralized with aqueous concentrated NH₄OH. The product was extracted with EtOAc and concentrated. Purification by chromatography and subsequent distillation (bp: 140° C. at 0.25 mbar) afforded the sub-title compound (2.24 g, 88%).

(d) 2-Amino-3-chlorophenol

To a cooled (0° C.) solution of 2-chloro-6-methoxyphenylamine (2 g, 12.7 mmol) in dry CH₂Cl₂ (20 mL) was added dropwise neat BBr₃ (14.8 mL, 50.8 mmol) and the mixture was stirred for 20 min at 0° C. and then for 20 min at ambient temperature. After cooling to −30° C. the reaction was quenched with MeOH (20mL), water was added and the product extracted with EtOAc. Concentration and recrystallization from EtOAc-petroleum ether afforded the sub-title compound (1.42 g, 78%).

(e) 4-Chloro-2-(2-chloro-5-nitrophenyl)benzoxazole

The sub-title compound was prepared from 2-amino-3-chlorophenol (see step (d) above) and 2-chloro-5-nitrobenzoyl chloride in accordance with Example 1 step (a).

(f) 4-Chloro-3-(4-chloro-benzoxazol-2-yl)phenylamine

The sub-title compound was prepared from 4-chloro-2-(2-chloro-5-nitrophenyl)benzoxazole (see step (e) above) in accordance with Example 34 step (b).

(g) N-[4-Chloro-3-(4-chlorobenzoxazol-2-yl)phenyl]-2-trifluoromethylbenzamide

The title compound was prepared from 4-chloro-3-(4-chlorobenzoxazol-2-yl)phenylamine (see step (f) above) and 2-trifluoromethylbenzoyl chloride in accordance with Example 1 step (c).

200 MHz ¹H-NMR (DMSO-d₆, ppm) δ 11.00 (1H, s) 8.65 (1H, d, J=2.6 Hz) 7.90 (1H, dd, J=8.8, 2.6 Hz) 7.87-7.72 (5H, m) 7.71 (1H, d, J=8.8 Hz) 7.56 (1H, dd, J=7.8, 1.4 Hz) 7.48 (1H, dd, J=7.8, 7.8 Hz).

### Example 48

### N-[3-(5-Bromobenzoxazol-2-yl)-4-chlorophenyl]-2,5-dichlorobenzenesulfonamide

To a cooled solution of 3-(5-bromobenzoxazol-2-yl)-4-chlorophenylamine (350 mg, 1.1 mmol; see Example 28 step (d)) in dry pyridine (15 mL) 2,5-dichlorobenzenesulfonyl chloride (322 mg, 1.31 mmol) was added. After stirring at room temperature for 4 h, the mixture was poured in water (50 mL) and extracted with EtOAc. The combined extracts were washed with water and brine and then dried over Na₂SO₄. Concentration under reduced pressure and purification by chromatography afforded the title compound (400 mg, 70%)

200 MHz ¹H-NMR (DMSO-d₆, ppm) δ 11.36 (1H, s) 8.14 (1H, d, J=1.9 Hz) 8.11 (1H, d, J=2.2 Hz) 7.91 (1H, d, J=2.6 Hz) 7.83 (1H, d, J=8.7 Hz) 7.77 (1H, dd, J=8.5, 2.2 Hz) 7.71 (1H, d, J=8.5 Hz) 7.65 (1H, dd, J=8.7, 1.9 Hz) 7.63 (1H, d, J=8.8 Hz) 7.36 (1H, dd, J=8.8, 2.6 Hz).

### Example 49

### 3.5-Dichloro-N-[3-(5-methylbenzoxazol-2-yl)phenyl]benzenesulfonamide

The title compound was prepared from 3(5-methylbenzoxazol-2-yl)phenylamine (see Example 1, step (b)) and 3,5-dichlorobenzenesulfonyl chloride in accordance with Example 48.

200 MHz ¹H-NMR (DMSO-d₆, ppm) δ 10.8 (1H, s) 7.95-7.86 (3H, m) 7.75 (2H, d, J=2.0 Hz) 7.64 (1H, d, J=8.4 Hz) 7.59-7.58 (1H, m) 7.51 (1H, dd, J=8.0, 8.0 Hz) 7.34 (1H, ddd, J=8.0, 2.2, 1.2 Hz) 7.23 (1H, dd, J=8.4, 1.2 Hz) 2.42 (3H, s).

### Example 50

### 3-Chloro-N-[4-chloro-3-(5-methylbenzoxazol-2-yl)phenyl]-2-methylbenzenesulfonamide

The title compound was prepared from 4-chloro-3-(5-methylbenzoxazol-2-yl)phenylamine (see Example 34, step (b)) and 3-chloro-2-methylbenzenesulfonyl chloride in accordance with Example 48.

200 MHz ¹H-NMR (DMSO-d₆, ppm) δ 11.10 (1H, s) 7.93 (1H, dd, J=8.0, 1.2 Hz) 7.86 (1H, d, J=2.8 Hz) 7.75-7.64 (3H, m) 7.56 (1H, d, J=8.8 Hz) 7.42 (1H, dd, J=8.0, 8.0 Hz) 7.30-7.24 (2H, m) 2.65 (3H, s) 2.43 (3H, s).

### Example 51

The following compounds were tested in the biological test described above and were found to exhibit 50% inhibition of mPGES-1 at a concentration of 10 μM or below:
2,4-dichloro-N-[2-methyl-5-(5,7-dimethylbenzoxazol-2-yl)phenyl]benzamide
2,5-dichloro-N-[3-(5,6-dimethylbemoxazol-2-yl)phenyl]benzamide;
2-chloro-N-[4-chloro-3-(5-chlorobenzoxazol-2-yl)phenyl]benzamide;
N-[4-chloro-3-(5-chlorobenzoxazol-2-yl)phenyl]-2-nitrobenzamide;
2-bromo-N-[3-(5,7-dichlorobenzoxazol-2-yl)phenyl]benzamide;
2-chloro-N-[4-chloro-3-(5-chlorobenzoxazol-2-yl)phenyl]-4-nitrobenzamide;
N-[4-chloro-3-(5-isopropylbenzoxazol-2-yl)phenyl]-2-nitrobenzamide;
2-chloro-N-[4-chloro-3-(5-isopropylbenzoxazol-2yl)phenyl]benzamide;
N-[4-chloro-3-(5-isopropylbenzoxazol-2-yl)phenyl]-4-methoxy-3-nitrobenzamide
2,5-dichloro-N-[3-(5-isopropylbenzoxaol-2-yl)phenyl]benzamide;
N-[4-chloro-3-(5-isopropylbenzoxazol-2-yl)phenyl]nicotinamide; and
2-chloro-N-[3-(5-methylbenzoxazol-2-yl)phenyl]benzamide.

### Example 52

Title compounds of Examples 1 to 50 were also tested in the biological test described above and were found to exhibit 50% inhibition of mPGES-1 at a concentration of 10 μM or below. For example, the following representative compounds of the examples exhibited the following IC₅₀ values:
Example 3: 1900 nM
Example 17: 1300 nM
Example 20: 2300 nM
Example 21: 1500 nM
Example 23: 2600 nM

1. A method for treating a disease in which inhibition or modulation of the activity of a member of the MAPEG family is desired and/or required which comprises administering to a subject in need of such inhibition or modulation an effective amount of a compound of formula I, 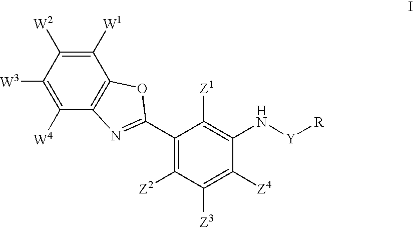
wherein
R represents aryl or heteroaryl, both of which are optionally substituted by one or more substituents selected from X¹;
Y represents —C(O)— or —S(O)₂—;
W¹ to W⁴ and Z¹ to Z⁴ independently represent hydrogen or a substituent selected from X²;
X¹ and X² independently represent halo, —R^{3a}, —CN, —C(O)R^{3b}, —C(O)OR^{3c},
—C(O)N(R^{4a})R^{5a}, —N(R^{4b})R^{5b}, —N(R^{3d})C(O)R^{4c}, —N(R^{3e})C(O)N(R^{4d})R^{5d},
—N(R^{3f})C(O)OR^{4e}, —N₃, —NO₂, —N(R^{3g})S(O)₂N(R^{4f})R^{5f}, —OR^{3h}, —OC(O)N(R^{4g})R^{5g},
—OS(O)₂R³ⁱ, —S(O)ₘR^{3j}, —N(R^{3k})S(O)₂R^{3m}, —OC(O)R³ⁿ, —OC(O)OR^{3p} or
—S(O)₂N(R^{4h})R^{5h};
m represents 0, 1 or 2;
R^{3b}, R^{3d} to R^{3h}, R^{3k}, R³ⁿ, R^{4a} to R^{4h}, R^{5a}, R^{5b}, R^{5d} and R^{5f} to R^{5h} independently represent H or R^{3a}; or
any of the pairs R^{4a} and R^{5a}, R^{4b} and R^{5b}, R^{4d} and R^{5d}, R^{4f} and R^{5f}, R^{4g} and R^{5g} or R^{4h} and R^{5h} may be linked together to form a 3- to 6-membered ring, which ring optionally contains a further heteroatom in addition to the nitrogen atom to which these substituents are necessarily attached, and which ring is optionally substituted by F, Cl, ═O or R^{3a};
R^{3c}, R³ⁱ, R^{3j}, R^{3m} and R^{3p} independently represent R^{3a};
R^{3a} represents C₁₋₆ alkyl optionally substituted by one or more substituents selected from F, Cl, ═O, —OR^{6a} or —N(R^{6b})R^{7b};
R^{6a} and R^{6b} independently represent H or C₁₋₆ alkyl optionally substituted by one or more substituents selected from F, Cl, ═O, —O^{8a}, —N(R^{9a})R^{10a} or —S(O)₂-G¹;
R^{7b} represents H, —S(O)₂CH₃, —S(O)₂CF₃ or C₁₋₆ alkyl optionally substituted by one or more substituents selected from F, Cl, ═O, —OR^{11a}, —N(R^{12a})R^{13a} or —S(O)₂-G²; or R^{6b} and
R^{7b} may be linked together to form a 3- to 6-membered ring, which ring optionally contains a further heteroatom in addition to the nitrogen atom to which these substituents are necessarily attached, and which ring is optionally substituted by F, Cl, ═O or C₁₋₃ alkyl optionally substituted by one or more fluoro atoms;
G¹ and G² independently represent —CH₃, —CF₃ or —N(R^{14a})R^{15a};
R^{8a} and R^{11a} independently represent H, —CH₃, —CH₂CH₃ or —CF₃;
R^{9a}, R^{10a}, R^{12a}, R^{13a}, R^{14a} and R^{15a} independently represent H, —CH₃ or —CH₂CH₃, or a pharmaceutically acceptable salt thereof.

2. A method as claimed in claim 1, wherein Y represents —C(O)—.

3. A method as claimed in claim 2, wherein when any of the pairs R^{4a} and R^{5a}, R^{4b} and R^{5b}, R^{4d} and R^{5d}, R^{4f} and R^{5f}, R^{4g} and R^{5g} or R^{4h} and R^{5h} are linked together, they together form a 3- to 6-membered ring, which ring optionally contains a further heteroatom in addition to the nitrogen atom to which these substituents are necessarily attached, and which ring is optionally substituted by ═O or R^{3a}.

4. A method as claimed in claim 3, wherein R^{3a} represents C₁₋₆ alkyl optionally substituted by one or more substituents selected from F, Cl, —OCH₃, —OCH₂CH₃ or —OCF₃.

5. A method as claimed in claim 4, wherein at least two of W¹ to W⁴ represents hydrogen.

6. A method as claimed in claim 5, wherein at least two of Z¹ to Z⁴ represents hydrogen.

7. A method as claimed in claim 6, wherein R is substituted with less than four substituents.

8. A method as claimed in claim 7, wherein X¹ and X² independently represent halo, —NO₂, —R^{3a} or —OR^{3h}.

9. A method as claimed in claim 8, wherein R^{3a} represents C₁₋₅ alkyl optionally substituted by one or more fluoro atoms.

10. A method as claimed in claim 9, wherein, when X¹ or X² represents R^{3a}, then R^{3a} represents t-butyl, t-pentyl, methyl, isopropyl or trifluoromethyl.

11. A method as claimed in claim 10, wherein, when R^{3h} represents R^{3a}, then R^{3a} represents cyclopentyl, difluoromethyl, ethyl, isopropyl, cyclopropyl, methyl or trifluoromethyl.

12. A method as claimed in claim 11, wherein W¹ to W⁴ independently represent H or a substituent selected from bromo, butyl, chloro, methyl and isopropyl.

13. A method as claimed in claim 12, wherein Z¹ to Z⁴ independently represent H or a substituent selected from fluoro, —OR^{3h}, —N(R^{4b})R^{5b}, chloro and methyl.

14. A method as claimed in claim 13, wherein when any one of Z¹ to Z⁴ represents —OR^{3h}, then R^{3h} represents H or C₁₋₅ alkyl.

15. A method as claimed in claim 14, wherein when any one of Z¹ to Z⁴ represents —N(R^{4b})R^{5b}, then R^{4b} and R^{5b} are independently selected from H or C₁₋₂ alkyl or, R^{4b} and R^{5b} are linked together with the nitrogen atom to which they are attached to form a pyrrolidinyl ring.

16. A method as claimed in claim 15, wherein R represents an optionally substituted phenyl, naphthyl, pyrrolyl, furanyl, thienyl, pyrazolyl, imidazolyl, oxazolyl, isoxazolyl, thiazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, or benzodioxanyl, group.

17. A method as claimed in claim 16, wherein R represents optionally substituted pyridyl or phenyl.

18. A method as claimed in claim 17 wherein, when R represents substituted phenyl, then the substituents are selected from —NH₂, chloro, fluoro, bromo, —NO₂, methyl, trifluoromethyl, methoxy and trifluoromethoxy.

19. A method as claimed in claim 17, wherein, when R represents substituted pyridyl, then the substituents are selected from fluoro, chloro and trifluoromethyl.

20. A method as claimed in claim 1 or claim 18, wherein R is phenyl, substituted in the ortho position relative to the point of attachment of the R group to the —C(O)— group in the compound of formula I.

21. A compound of formula I as defined in claim 1, or a pharmaceutically-acceptable salt thereof, for use as a pharmaceutical, provided that when Y represents —C(O)—, W¹, Z¹ and Z³ all represent hydrogen, and:
(A) W², W³ and W⁴ all represent H, then:
(i) when Z² represents H and Z⁴ represents —NH₂, then R does not represent 4-(aminoacetyl)phenyl;
(ii) when Z² represents chloro and Z⁴ represents H, then R does not represent 4-ethoxy-3-nitrophenyl, 3,4,5-trimethoxyphenyl, 3,5-dimethoxyphenyl or 2-methyl-3-nitrophenyl;
(iii) when Z² and Z⁴ both represent H, then R does not represent 3,5-dinitro-4-methylphenyl;
(B) W² and W⁴ both represent H, then:
(i) when W³ represents chloro, Z² represents H and Z⁴ represents —CH₃, then R does not represent 2-methoxyphenyl;
(ii) when W³ represents —CH₃, Z² represents chloro and Z⁴ represents H, then R does not represent unsubstituted phenyl;
(C) W², W³, Z² and Z⁴ all represent H and W⁴ represents —CH₃, then R does not represent 3-methylphenyl;
(D) W³, W⁴ and Z⁴ all represent H, then:
(i) when W² represents bromo and Z² represents H, then R does not represent unsubstituted phenyl;
(ii) when W² represents —CH₃ and Z² represents chloro, then R does not represent unsubstituted 3-pyridyl;
(E) W² and W³ both represent —CH₃, W⁴ represents H, Z² represents chloro and Z⁴ represents H, then R does not represent unsubstituted phenyl.

22. A pharmaceutical formulation including a compound as defined in claim 21, or a pharmaceutically acceptable salt thereof, in admixture with a pharmaceutically acceptable adjuvant, diluent or carrier.

23. A compound of formula I as defined in claim 1, in which Z³ represents a substituent selected from X², or a pharmaceutically-acceptable salt thereof, provided that when W¹ to W⁴, Z¹, Z² and Z⁴ all represent hydrogen and Z³ represents —CH₃, then R does not represent 4-ethoxyphenyl.

24. A compound of formula I as defined in claim 1, but in which any two of Z¹ to Z⁴ represent a substituent selected from X², or a pharmaceutically-acceptable salt thereof.

25. A compound of formula I as defined in claim 1, but in which Y represents —S(O)₂—, or a pharmaceutically-acceptable salt thereof, provided that when W⁴ represents H, Z³ represents H, and:
(A) W¹, W², Z² and Z⁴ all represent H, W³ represents methyl, and:
(i) Z¹ represents H, then R does not represent unsubstituted phenyl, 4-methylphenyl, 4-(aminoacetyl)phenyl or 4-chlorophenyl;
(ii) Z¹ represents methyl, then R does not represent unsubstituted phenyl;
(B) W¹, W², W³, Z¹ and Z⁴ all represent H, and:
(i) Z² represents —OH or Cl, then R does not represent unsubstituted phenyl;
(ii) Z² represents H, then R does not represent unsubstituted phenyl, 4-chlorophenyl, 4-nitrophenyl, 4-(aminoacetyl)phenyl or 4-methylphenyl;
(C) Z⁴ represents methyl and Z¹ and Z² both represent H, and:
(i) W² represents H and W¹ and W³ both represent methyl; or
(ii) W² represents methyl and W¹ and W³ both represent H, then (in both cases) R does not represent 2-chloro-5-nitrophenyl;
(D) W², Z¹ and Z² all represent H, and:
(i) W¹ represents H, W³ represents ethyl or chloro and Z⁴ represents methyl or H; or
(ii) W¹ and W³ represent chloro and Z⁴ represents methyl, then (in both cases) R does not represent unsubstituted phenyl.

26. A method as claimed in claim 1, wherein the member of the MAPEG family is microsomal prostaglandin E synthase-1, leukotriene C₄ and/or 5-lipoxygenase-activating protein.

27. A method as claimed in claim 26, wherein the member of the MAPEG family is microsomal prostaglandin E synthase-1.

28. A method as claimed in claim 26 or claim 27, wherein the disease is inflammation.

29. A method as claimed in claim 26, wherein the disease is asthma, chronic obstructive pulmonary disease, pulmonary fibrosis, inflammatory bowel disease, irritable bowel syndrome, inflammatory pain, fever, migraine, headache, low back pain, fibromyalgia, a myofascial disorder, a viral infection, a bacterial infection, a fungal infection, dysmenorrhea, a burn, a surgical or dental procedure, a malignancy, hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, a neurodegenerative disorder, an autoimmune disease, an allergic disorder, rhinitis, an ulcer, coronary heart disease, sarcoidosis, any other disease with an inflammatory component, osteoporosis, osteoarthritis, Paget's disease or a periodontal disease.

30. A method of treatment of a disease in which inhibition of the activity of a member of the MAPEG family is desired and/or required, which method comprises administration of a therapeutically effective amount of a compound as defined in claim 1 or claim 20, or a pharmaceutically-acceptable salt thereof, to a patient suffering from, or susceptible to, such a condition.

31. A method as claimed in claim 30, wherein the member of the MAPEG family is microsomal prostaglandin E synthase-1, leukotriene C₄ and/or 5-lipoxygenase-activating protein.

32. A method as claimed in claim 31, wherein the member of the MAPEG family is microsomal prostaglandin E synthase-1.

33. A combination product comprising:
(A) a compound of formula I, as defined in claim 1 or claim 20, or a pharmaceutically-acceptable salt thereof; and
(B) another therapeutic agent that is useful in the treatment of inflammation, wherein each of components (A) and (B) is formulated in admixture with a pharmaceutically-acceptable adjuvant, diluent or carrier.

34. A combination product as claimed in claim 33 which comprises a pharmaceutical formulation including a compound of formula I, or a pharmaceutically-acceptable salt thereof, another therapeutic agent that is useful in the treatment of inflammation, and a pharmaceutically-acceptable adjuvant, diluent or carrier.

35. A combination product as claimed in claim 33 which comprises a kit of parts comprising components:
(a) a pharmaceutical formulation including a compound of formula I, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

36. A process for the preparation of a compound of formula I as defined in claim 21, which comprises:
(i) reaction of a compound of formula II, 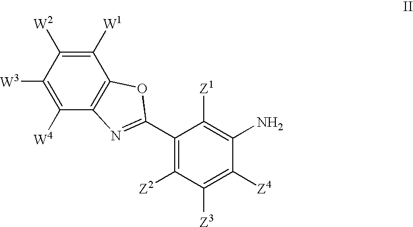
wherein W¹ to W⁴ and Z¹ to Z⁴ are as defined in claim 1, with a compound of formula III,
R—Y—OH III
wherein R and Y are as defined in claim 1; or
(ii) reaction of a compound of formula IV, 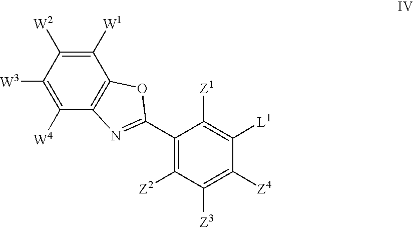
wherein L¹ represents a suitable leaving group, and W¹ to W⁴ and Z¹ to Z⁴ are as defined in claim 1, with a compound of formula V,
H₂N—Y—R V
wherein R and Y are as defined in claim 1.

37. A process for the preparation of a pharmaceutical formulation as defined in claim 22, which process comprises bringing into association a compound of formula I, as defined in claim 21, or a pharmaceutically acceptable salt thereof with a pharmaceutically-acceptable adjuvant, diluent or carrier.

38. A process for the preparation of a combination product as defined in claim 33, which process comprises bringing into association a compound of formula I, as defined in claim 1, or a pharmaceutically acceptable salt thereof with another therapeutic agent that is useful in the treatment of inflammation, and a pharmaceutically-acceptable adjuvant, diluent or carrier.
